(19) United States
(12) Patent Application Publication
(10) Pub. No.: US 2013/0295081 A1
(43) Pub. Date: Nov. 7, 2013

(54) Polyurethane Composite for Wound Healing and Methods Thereof

(71) Applicant: Vanderbilt University, Nashville, TN (US)

(72) Inventors: Scott A. Guelcher, Thompson Station, TN (US); Andrea Hafeman, Hillsborough, CA (US); Jeffrey Davidson, Nashville, TN (US); Lillian M. Nanney, Nashville, TN (US); Elizabeth Adolph, Nashville, TN (US)

(21) Appl. No.: 13/864,224

(22) Filed: Apr. 16, 2013

Related U.S. Application Data
(63) Continuation-in-part of application No. 12/608,850, filed on Oct. 29, 2009.
(60) Provisional application No. 61/624,887, filed on Apr. 16, 2012.
(60) Provisional application No. 61/242,758, filed on Sep. 15, 2009.
(60) Provisional application No. 61/120,836, filed on Dec. 8, 2008.
(60) Provisional application No. 61/109,892, filed on Oct. 30, 2008.

(51) Int. Cl. A61L 26/00; A61L 15/26

(52) U.S. Cl. CPC A61L 26/0019; A61L 26/0066; A61L 15/26
USPC 424/130.1; 523/113; 514/788; 514/561; 514/1.1; 514/20.9; 514/169; 514/23; 424/562; 424/94.1; 424/93.7; 514/9.3; 514/44 R; 424/93.1; 521/170

(57) ABSTRACT

The presently-disclosed subject matter includes polyurethane composites that include tissue component(s), as well as methods of making such composites and uses thereof. The polyurethane component can comprise a polyisocyanate prepolymer and a polyol. The tissue component can be a polysaccharide. Exemplary composites can be moldable and/or injectable, and can cure into a porous composite that provides mechanical strength and/or supports the in-growth of cells. Inventive composites have the advantage of being able to fill irregularly shaped areas, voids, or the like. Exemplary composites can be used for treating wounds.

### RELATED APPLICATIONS

This application claims the benefit of U.S. Provisional Application Ser. No. 61/624,887, filed Apr. 16, 2012, and is a continuation-in-part of U.S. patent application Ser. No. 12/608,850, filed Oct. 29, 2009, which claims the benefit of U.S. Provisional Application Ser. No. 61/242,758, filed Sep. 15, 2009, U.S. Provisional Application Ser. No. 61/120,836, filed Dec. 8, 2008, and U.S. Provisional Application Ser. No. 61/109,892, filed Oct. 30, 2008, the entire disclosures of which are incorporated herein by this reference.

### GOVERNMENT SUPPORT

This invention was made with government support under Grant Nos. AG06528 and AR056138 awarded by the National Institutes of Health, Grant No. W81XWH-07-1-0211 awarded by the Department of Defense, and the Department of Veterans Affairs. The US government has certain rights in the invention.

### FIELD OF THE INVENTION

The present invention generally relates to composites and methods for use the same. More specifically, certain embodiments of the present invention relate to injectable polyurethane composites for wound repair and regeneration, and that may further comprise polysaccharides and optionally other substances.

### BACKGROUND OF THE INVENTION

Wound healing is a universal problem, particularly given the increases in immobile aging, diabetic amputees, paralyzed patients afflicted with large chronic wounds and fistulas, and trauma victims with large cutaneous defects. These well known problems indicate a need for the development of injectable biomaterials to promote restoration of tissue integrity. Such scaffolds could offer new options for both cutaneous and fascial indications while adding options for site-specific customization. Furthermore, a biomaterial that is applied as a liquid and cures in situ can flow to fill the contours of irregularly shaped defects that may not conform to a preformed implant. Maximizing the contact surface area between the material and surrounding tissue should enhance cellular infiltration and integration of the scaffold.

Natural and synthetic polymers including collagen, chitosan, fibrin, and poly(lactic-co-glycolic acid) are currently used in cutaneous wound healing in the form of hydrogels, sheets, sponges, and electrospun scaffolds.²⁴ These polymers are advantageous due to their biocompatibility and biodegradability, but they present potential drawbacks such as low modulus and strength, small pore size, and low porosity.¹² Specifically, the microstructure of synthetic hydrogels is typically smaller than the average size of cellular populations (5-15 μm)¹², thus requiring resorption or displacement of the matrix by cells that results in slow infiltration of the scaffold. Low mechanical properties result in undesirable outcomes such as contraction and scarring. Hydrogels also lack the tough, elastomeric properties of thermoplastic polymers that are appropriate for cutaneous applications.

Scaffolds with >90% porosity are desirable because they can easily support infiltration of new tissue and transport of nutrients and waste.²⁵ A studies have reported optimal pore sizes for fibroblast infiltration and new tissue ingrowth ranging from 90-360 μm²⁶, slow infiltration and vasularization with small pores and/or low porosity,²⁴ and the viability of seeded fibroblasts may be highest for pore size <160 μm.²⁷′²⁸ Another study resulted in low viability of fibroblasts in scaffolds with pores ranging from 50-80 μm compared to scaffolds with larger pores.²⁵

Nanofibrous scaffolds have potential for use in cutaneous wound healing because they mimic the structure and function of natural ECM.²⁴ Despite their small pores, their high surface area to volume ratio results in excellent permeability for oxygen and nutrients.²⁴ Delivery of recombinant human platelet-derived growth factor (rhPDGF) from nanofibrous PLGA scaffolds has been reported to enhance wound healing in rats²⁹, and another study has examined the use of bioactive poly-N-acetyl-glucosamine nanofibrous membranes in cutaneous wounds in diabetic mice.³⁰ The nanofibers enhanced keratinocyte migration, cell proliferation, and angiogenesis compared to a cellulose control.³⁰ However, pre-formed implants such as nanofibrous scaffolds cannot be injected, and thus cannot fill and conform to deep tissue defects.

Analyzing the shortcomings of prior materials, several requirements may be identified as being important to the success of injectable biomaterials, including flowability for a sufficient time (working time) to enable injection, and curing within minutes of injection (setting time) to avoid long surgical procedures. Working and setting times are therefore highly relevant in determining whether a product is adequate for clinical, emergency, or other applications. Injected materials should not have adverse effects on surrounding host tissue due to the reactivity of specific components or to the release of heat through a reaction exotherm.² The viscosity of the injected material may be high enough to be retained at the injection site and to minimize extravasation into surrounding tissues where it may have an adverse effect.³ The reproducibility of properties such as porosity, degradation, and setting time in clinical environments is also a significant challenge. Injectable porous biomaterials must have a suitable pore structure for cell migration, nutrient exchange, and tissue ingrowth.⁴

Therefore, while progress has been made in the development of biocompatible and biodegradable polymers, it remains desirable to develop biocompatible and biodegradable polymers that, inter alia, exhibit highly porous structures, have work and set times that are desirable for wound healing applications, adapt to irregular wound shapes and thicknesses, support cellular infiltration, are nontoxic, and may deliver biologics and other substances to a would site. Furthermore, there remains a long-felt but unmet need for methods of synthesizing such polymers, implantable devices comprising such polymers, and methods of using such polymers.

### BRIEF SUMMARY OF THE INVENTION

Embodiments of the present invention relate to, without limitation, injectable polyurethane (PUR) composite scaffolds that may incorporate polysaccharides and optionally biologics or synthetically derived analogs. Embodiments of the injectable PUR are capable of forming in situ and conforming three dimensionally to the area applied, including cutaneous wounds. Embodiments of the present invention are capable of meeting long felt but unmet needs, particularly in the field of wound healing, by providing nontoxic, biodegradable, biocompatible, and porous scaffolds with work and set times that are practical for wound healing applications. The present invention also relates to methods for synthesizing and using PUR scaffolds, including in wound healing applications. It is understood that the present invention may comprise additional elements, including those that are delivered to a wound site via the scaffold.

In certain embodiments the PUR scaffold of the present invention is of a viscosity that allows the scaffold to be injected and remain at the injection site during the setting time while minimizing extravasation into surrounding tissues. In some embodiments the PUR scaffold is injected onto or into a wound site and is allowed to set. Certain embodiments are advantageous when compared to prior methods of wound healing because the injectable PUR may act as a void filler to fill, cover, and heal irregularly shaped wounds, including cutaneous wounds. In other embodiments the PUR composite can be molded, and then the molded composite can be placed on a wound site.

Certain embodiments of the present invention are synthesized by combining lysine triisocyanate (LTI), poly(ethylene glycol), a polyester triol, tissue component, water, a catalyst, a blowing catalyst, and/or a pore opener. In some embodiments the itssue component is a polysaccharide. Embodiments may comprise all of or only some of the previously stated materials, and appropriate substitutions may be made for materials without straying from the scope of the invention. Embodiments may comprise various types of polysaccharides, including hyaluronic acid (HA), carboxylmethyl cellulose (CMC), and/or sucrose.

Porosity of embodiments of the present invention may vary from 30-70% and pore size may range from about 100-700 μm. Porosity and pore size may be optimized, possibly by adjusting proportions of ingredients, so as to maximize cellular infiltration as well as other physical attributes of the PUR scaffolds.

Embodiments of the present invention meet the unmet need of providing a scaffold that may delay wound contraction, enhance cellular proliferation, and reduce alignment of scar collagen, thereby enhancing the wound healing process and minimizing undesirable long-term effects, such as scarring.

Embodiments of the present invention meet the unmet need of a product that exhibits biocompatibility, ease of use, clinically relevant working and setting times, support of cellular infiltration, positive impact on matrix remodeling, and the potential to deliver biologics.

### DEFINITIONS

The term “bioactive agent” is used herein to refer to compounds or entities that alter, promote, speed, prolong, inhibit, activate, or otherwise affect biological or chemical events in a subject (e.g., a human or mammalian). For example, bioactive agents may include, but are not limited to adipogenic, adipoinductive, and adipoconductive agents, vasculogenic, vasculoinductive, and vasculoconductive agents, chondrogenic, chondroinductive, and chondroconductive agents anti-HfV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of ON A, RNA, or protein synthesis, anti-hypertensives, analgesics, antipyretics, steroidal and non-steroidal anti-inflammatory agents, anti-angiogenic factors, angiogenic factors, anti-secretory factors, anticoagulants and/or antithrombotic agents, local anesthetics, reactive oxygen species inhibitors, chelating agents, ophthalmics, prostaglandins, anti-depressants, anti-psychotics, targeting agents, chemotactic factors, receptors, neurotransmitters, proteins, cell response modifiers, cells, peptides, polynucleotides, viruses and vaccines. In certain embodiments, the bioactive agent is a drug. In certain embodiments, the bioactive agent is a small molecule.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-251National Formulary-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21 C.F.R. §§330.5, 331 through 361, and 440 through 460, and drugs for veterinary use listed by the FDA under 21 C.F.R. §§500 through 589, all of which are incorporated herein by reference, are also considered acceptable for use in accordance with the present invention.

The terms, “biodegradable”, “biodegradable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “carbohydrate” as used herein, refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′ fluororibose, deoxyribose, and hexose). Carbohydrates may exist in many different forms, for example, conformers, cyclic forms, acyclic forms, stereo isomers, tautomers, anomers, and isomers.

The term “composite” as used herein, is used to refer to a unified combination of two or more distinct materials. The composite may be homogeneous or heterogeneous. For example, a composite may be a combination of tissue component (which includes a tissue subcomponent or particle) and a polymer; or a combination of tissue component, polymers and antibiotics; or the polymer and an excipient molecule or other structure. In certain embodiments, the composite has a particular orientation.

The term “flowable polymer material” as used herein, refers to a flow able composition including one or more of monomers, pre-polymers, oligomers, low molecular weight polymers, uncross-linked polymers, partially cross-linked polymers, partially polymerized polymers, polymers, or combinations thereof that have been rendered formable. One skilled in the art will recognize that a flowable polymer material need not be a polymer but may be polymerizable. In some embodiments, flowable polymer materials include polymers that have been heated past their glass transition or melting point. Alternatively or in addition, a flowable polymer material may include partially polymerized polymer, telechelic polymer, or prepolymer. A pre-polymer is a low molecular weight oligomer typically produced through step growth polymerization. The pre-polymer is formed with an excess of one of the components to produce molecules that are all terminated with the same group. For example, a diol and an excess of a diisocyanate may be polymerized to produce isocyanate terminated prepolymer that may be combined with a diol to form a polyurethane. Alternatively or in addition, a flowable polymer material may be a polymer material/solvent mixture that sets when the solvent is removed.

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The term “tissue conductive” as used herein, refers to the ability of a substance or material to provide surfaces which are receptive to the growth of new tissue.

The term “tissue-genic” as used herein, refers to the ability of a substance or material that can induce or accelerate new or remodeled tissue formation.

The term “tissue inductive” as used herein, refers to the quality of being able to recruit cells (e.g., fibroblasts, endothelial, mesenchymal stem cells) from the host that have the potential to stimulate new tissue formation. In general, tissue-inductive materials are capable of inducing heterotopic tissue formation in dissimilar terminally differentiated soft tissues (e.g., muscle).

The term “STimplant” or “soft tissue-implant” is used herein in its broadest sense and is not intended to be limited to any particular shapes, sizes, configurations, compositions, or applications. STimplant refers to any device or material for implantation that aids or augments tissue formation or healing. STimplants are often applied at a tissue defect site, e.g., one resulting from injury, defect brought about during the course of surgery, infection, malignancy, inflammation, or developmental malformation. STimplants can be used in a variety of surgical procedures such as the repair of simple and complex tissue defects from tumor removal as in mastectomy or sarcoma excions or traumatic such as liver laceration or facial soft tissue defects or chronic disease states, etc.

The terms “polynucleotide”, “nucleic acid”, or “oligonucleotide” as used herein, refer to a polymer of nucleotides. The terms “polynucleotide”, “nucleic acid”, and “oligonucleotide”, may be used interchangeably. Typically, a polynucleotide comprises at least three nucleotides. DNAs and RNAs are exemplary polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as RNAi, siRNA, or shRNA.

The terms “polypeptide”, “peptide”, or “protein” as used herein, include a string of at least three amino acids linked together by peptide bonds. The terms “polypeptide”, “peptide”, and “protein”, may be used interchangeably. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater halflife in vivo). These modifications may include cyclization of the peptide, the incorporation of D-amino acids, etc. None of the modifications should substantially interfere with the desired biological activity of the peptide.

The terms “polysaccharide” or “oligosaccharide” as used herein, refer to any polymer or oligomer of carbohydrate residues. Polymers or oligomers may consist of anywhere from two to hundreds to thousands of sugar units or more. “Oligosaccharide” generally refers to a relatively low molecular weight polymer, while “polysaccharide” typically refers to a higher molecular weight polymer. Polysaccharides may be purified from natural sources such as human, animal (e.g., hyaluronic acid), or other species (e.g., chitosan) and plants (e.g., alginate) or may be synthesized de novo in the laboratory. Polysaccharides isolated from natural sources may be modified chemically to change their chemical or physical properties (e.g., reduced, oxidized, phosphorylated, cross-linked). Carbohydrate polymers or oligomers may include natural sugars (e.g., glucose, fructose, galactose, sucrose, mannose, arabinose, ribose, xylose, etc.) and/or modified sugars (e.g., 2′-fluororibose, 2′ deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, poly(dextrose), and fructose. In some embodiments, glycosaminoglycans are considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

The term “porogen” as used herein, refers to a chemical compound that may be part of the inventive composite and upon implantation/injection or prior to implantation/injection diffuses, dissolves, and/or degrades to leave a pore in the osteoimplant composite. A porogen may be introduced into the composite during manufacture, during preparation of the composite (e.g., in the operating room), or after implantation/injection. A porogen essentially reserves space in the composite while the composite is being molded but once the composite is implanted the porogen diffuses, dissolves, or degrades, thereby inducing porosity into the composite. In this way porogens provide latent pores. In certain embodiments, the porogen may be leached out of the composite before implantation/injection. This resulting porosity of the implant generated during manufacture or after implantation/injection (i.e., “latent porosity”) is thought to allow infiltration by cells, tissue formation, tissue remodeling, osteoinduction, osteoconduction, and/or faster degradation of the osteoimplant. A porogen may be a gas (e.g., carbon dioxide, nitrogen, or other inert gas), liquid (e.g., water, biological fluid), or solid. Porogens are typically water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade quickly under physiological conditions. Exemplary polymers include polyethylene glycol, poly(vinylpyrollidone), pullulan, poly(glycolide), poly(lactide), poly(lactide-co-glycolide), other polyesters, and starches. In certain embodiments, tissue and/or sub components or a synthetic analog excipient utilized in provided composites or compositions may act as porogens.

Some embodiments, porogens may refer to a blowing agent (i.e., an agent that participates in a chemical reaction to generate a gas). Water may act as such a blowing agent or porogen.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (E), defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation/injection. It will be appreciated by these of ordinary skill in the art that the porosity of a provided composite or composition may change over time, in some embodiments, after implantation/injection (e.g., after leaching of a porogen, when the porogen degrades either by dissolution, hydrolytic, or cell-mediated degradation via tissue remodeling mononuclear/multi-nucleated cell resorbing a graft tissue, etc.). For the purpose of the present disclosure, implantation/injection may be considered to be “time zero” (To). In some embodiments, the present invention provides composites and/or compositions having a porosity of at least about 30%, at least about 40%, at least about 50%, at least about 60%, at least about 70%, at least about 80%, at least about 90% or more than 90%, at time zero. In certain embodiments, pre-molded composites and/or compositions may have a porosity of at least about 30%, at least about 40%, at least about 50%, at least about 60%, at least about 70%, at least about 80%, at least about 90% or more than 90%, at time zero. In certain embodiments, injectable composites and/or compositions may have a porosity of as low as 3% at time zero. In certain embodiments, injectable composites and/or compositions may cure in situ and have a porosity of at least about 30%, at least about 40%, at least about 50%, at least about 60%, at least about 70%, at least about 80%, at least about 90% or more than 90% after curing.

The term “remodeling” as used herein, describes the process by which native tissue, processed tissue allograft, whole tissue sections employed as grafts, and/or other tissues are replaced with new cell-containing host tissue by the action of local mononuclear and multinuclear cells. Remodeling also describes the process by which non-osseous native tissue and tissue grafts are removed and replaced with new, cell-containing tissue in vivo. Remodeling also describes how inorganic materials (e.g., calcium-phosphate materials, such as f3-tricalcium phosphate) is replaced with living tissue.

The term “setting time” as used herein, is approximated by the tack-free time (TFT), which is defined as the time at which the material could be touched with a spatula with no adhesion of the spatula to the foam. At the TFT, the wound could be closed without altering the properties of the material.

The term “shaped’ as used herein, is intended to characterize a material (e.g., composite) or a soft tissue-implant refers to a material or soft tissue-implant of a determined or regular form, 3-D conformation or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, tissues, portions of tissues, wedges, cylinders, threaded cylinders, and the like, as well as more complex geometric configurations.

The term “small molecule” as used herein, is used to refer to molecules, whether naturally-occurring or artificially created (e.g., via chemical synthesis), that have a relatively low molecular weight. In some embodiments, small molecules have a molecular wight of less than about 2,500 g/mol, for example, less than 1000 g/mol. In certain embodiments, small molecules are biologically active in that they produce a loacal or systemic effect in animals, such as mammals, e.g., humans. In certain embodiments, a small molecule is a drug. In certain embodiments, though not necessarily, a durg is one that has already been deemed safe and effective for use by an apporopriate governmental agency or body (e.g., the U.S. Food and Drug Administration).

The terms “subject” or “subject in need thereof” refer to a target of administration and/or treatment, which optionally displays symptoms related to a particular disease, injury, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

The term “transformation” as used herein, describes a process by which a material is romved from an implant site and replaced by host tissue after implantation. Transformation may be accomplished by a combination of processes, including but not limited to remodeling, degradation, resporption, and tissue growth and/or formation. Removal of the material may be cell-mediated or accomplished through chemical processes, such as dissolution and hydrolysis.

The term “wet compressive strength” as used herein, refers to the compressive strength of a soft tissue implant (STimplant) after being immersed in physiological saline (e.g., phosphate-buffered saline (PBS), water containing 0.9 g NaCIIIOO ml water, etc.) for a minimum of 12 hours (e.g., 24 hours). Compressive strength and modulus are well-known measurements of mechanical properties and is measured using the procedure described herein.

The term “working time” as used herein, is defined in the IS0991 7 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows. According to the present invention, the working time is measured by loading the syringe with the reactive composite and injecting <0.25 ml every 30 s. The working time is noted as the time at which the material was more difficult to inject, indicating a significant change in viscosity.

### BRIEF DESCRIPTION OF THE DRAWINGS

The following figures of embodiments and data obtained from embodiments are examples, rather than limitations, in which reference may indicate similar elements and in which:

FIG. 1 depicts chemical structures and reactivities, where (A) shows chemical structures of HA (above) and CMC (below), and (B) shows the determination of second-order rate constants for the reactions of polyester triol, HA, and CMC with LTI-PEG prepolymer (water data not shown: kw=600 g mol−1 min−1).

FIG. 2 shows data of rheological properties of injectable PUR scaffolds, where (A) shows data for a PUR scaffold, (B) shows data for a PUR+CMC scaffold, (C) shows data for a PUR+HA scaffold, and (D) shows temperature profiles during cure for PUR, PUR+15% CMC, and PUR+30% CMC scaffolds. The G-crossover points are considered to be the gel point and thus the working time of the foams.

FIG. 3 shows SEM images and data for the degradation of embodiments of LTI-PEG PUR scaffolds (arrows indicate HA particles), where (A.1) is a SEM image of a no-additive PUR scaffold, (A.2) and (A.3) are, respectively, low and high magnification SEM images of a PUR+HA scaffold with embedded HA particles, (A.4) is a SEM image of a PUR+HA that was foamed in a high-moisture environment, similar to that which would occur in vivo, and (B) is a chart of degradation of injectable PUR scaffolds in PBS at 37° C. (n=3).

FIG. 4 shows data of wounds from the blank, PUR+HA, and PUR+CMC treatment groups 7, 17, 26, and 35 days following surgery, where (A) shows a schematic summarizing measured wound dimensions using a representative image of PUR+HA at day 26, wherein wound gap (line 1), wound thickness (line 2), and percent re-epithelialization (sum of lines 3 and 4 divided by sum of lines 3, 4, and 5) are labeled, (B) shows wound thickness (mm), (C) shows wound length (mm), and (D) shows percentage of reepithelialization.

FIG. 5 shows data of immunohistochemical staining for Ki67 tissue sections from embodiments of blank, PUR+HA, and PUR+CMC treatment groups, where (A) shows Ki67 staining at days 7, 17, 26, and 35 following surgery indicating the level of cell proliferation within the wound bed, and (B) shows TUNEL staining at days 7, 17, and 35 following surgery to measure cell apoptosis in the wound site.

FIG. 6 shows images of tissue sections from blank, PUR+HA, and PUR+CMC treatment groups at days 17, 26, and 35 following surgery stained for α-smooth muscle actin (α-SMA), wherein remnants of PUR foam (F), blood vessels (B), and myofibroblasts (M) are indicated by arrows. Blood vessels that exhibit immunoreactivity for α-SMA are not labeled in the images. Scale bar=100 μm.

FIG. 7 shows images of tissue sections from blank, PUR+HA, and PUR+CMC treatment groups at days 17, 26, and 35 following surgery stained with picrosirius red and observed with polarized light microscopy, wherein remnants of PUR foam are labeled (F). Scale bar=200 μm.

FIG. 8 shows images of tissue sections from blank, PUR+HA, and PUR+CMC treatment groups at days 17, 26, and 35 following surgery stained for procollagen I, wherein remnants of PUR foam are labeled (F). Scale bar=100 μm.

FIG. 9 shows data of the number of procollagen I producing cells in each of the blank, PUR+HA, and PUR+CMC treatment groups at days 17, 26, and 35 days following surgery.

FIG. 10 shows SEM images of the surface of polyurethane composites.

FIG. 11 shows data of the air permeability of polyurethane composites comprising lysine triisocyanate-poly(ethylene glycol) prepolymers with and without treatment to inhibit skin formation.

FIG. 12 shows the chemical structures for A) 4-para-amino benzoic acid (PABA)-lactide-diethylene glycol diisocyanate (PLD), and B) 4-para-amino benzoic acid (PABA)-glycolide-diethylene glycol diisocyanate (PGD).

FIG. 13 shows data of the porosity of PLD and PGD composites that was measured via SEM and gravimetric analysis (GMA).

FIG. 14 shows SEM micrographs of PLD and PGD composites before and after leaching sugar, where A) shows PLD composite before leaching, B) shows PGD composite before leaching, C) shows PLD composite after leaching sugar for 4 days, and D) shows PGD composite after leaching sugar for 4 days.

FIG. 15 shows degradation data for PLD and PGD composites at 57° C. in PBS.

FIG. 16 shows elastic modulus data for dry and PBS soaked PLD and PGD composites. *p<0.05 for both dry and wet PLD samples; # p<0.05 for PLD wet samples only.

FIG. 17 shows ATR-FTIR spectra in the carbonyl region for PLD and PGD composites.

FIG. 18 shows differential scanning calorimetry spectra for PGD and PLD composites.

FIG. 19 shows histological sections for pig excisional wounds at 8 days that were treated with A) lysine triisocyanate-containing composites or B) no treatment (control).

FIG. 20 shows histological sections for pig excisional wounds at 8 days that were treated with polyurethane composites comprising 40 wt % sucrose.

FIG. 21 shows histological sections for pig excisional wounds at 8 days that were treated with polyurethane composites comprising 70 wt % sucrose.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this paper, in certain instances, the terms “foam”, “scaffold”, “composite”, “composition” and the like may be used interchangeably to refer to certain embodiments of the present invention.

The presently-disclosed subject matter includes a biocompatible and biodegradable polymer composites. The composition may comprise a polysaccharide and a polymer. Embodiments of the present invention include biocompatible and biodegradable polymeric composite foams. Embodiments may comprise polyurethane (PUR) composites that are preferably biodegradable on or within a living organism.

In certain embodiments these composites are injectable. Related embodiments of the present invention include methods and compositions for their preparation and the use of these composites for wound healing applications as kits for preparing and/or administering the respective embodiments.

Embodiments of PUR scaffolds may also serve as delivery vehicles for additives such as antibacterial, growth, and other factors. For instance, some embodiments comprise at least one biologically active molecule having at least one active hydrogen. Certain embodiments may also be designed to not be cytotoxic, have a minimal reaction exotherm to avoid necrosis of surrounding tissues, and/or achieving interconnected pores while retaining robust mechanical properties.

An embodiment of the present invention is an injectable, biodegradable, and/or bioresorbable polyurethane (PUR) foam with polysaccharides to promote and facilitate wound healing while minimizing scarring and other negative aspects associated with wound healing. Embodiments of the foams may be made by combining: (a) a prepolymer, (b) a hardener component, and (c) a tissue component (e.g., polysaccharide). The prepolymer may be a lysine triisocyanate (LTI)—poly(ethylene glycol) (PEG) prepolymer and may be flowable. The hardener component may comprise a polyester triol (polyol), which may be a poly(ε-caprolactone-co-glycolide-co-lactide)polyol and may be flowable, water, a catalyst, and a pore opener. In embodiments comprising a polysaccharide as the tissue component, the polysaccharide may be added to the reactive PUR, and may be chosen from hyaluronic acid (HA), 1,500-2,200-kDa glycosaminoglycan found in the extracellular matrix, carboxylmethyl cellulose (CMC), a plant-derived 90-kDa polysaccharide, sucrose, and the like. In certain embodiments, and without being bound by theory or mechanism, the addition of a tissue component (e.g., polysaccharide) controls the foaming of the PUR scaffold through absorption of excess moisture from the wound bed or site. The absence of polysaccharides or other alternative substances may lead to PUR scaffolds that potentially over-expand and form large voids in vivo. Any suitable polysaccharide or other tissue component that achieves desired results may be utilized in the present invention. Preferably, all of the components used in embodiments of the present invention are nontoxic, alone or in combination.

Embodiments of PUR scaffolds of the present invention provide a significant improvement over current tissue graft and scaffold treatments. They may be both biodegradable and resorbable, allowing for minimized total surgery time and invasiveness for patients. A benefit of the reactive liquid molding synthesis of embodiments of PUR scaffolds is that it may allow them to be injectable and therefore minimally invasive during implantation. In addition, embodiments of the present invention may expand to fill the contours of the wound site, which may be large or irregularly shaped, enhancing tissue-scaffold contact and fixation.

Tissue Component

In certain embodiments of the present invention, an additional component may be referred to as a tissue component, and may include a tissue-derived material, an inorganic material, a synthetic analog or animal or plant species tissue component, a tissue substitute material, a composite material, or any combinations thereof. As discussed below a tissue component may refer to autologous, allogenic, xenogenic tissue or a tissue subcomponent such as, but not limited to, a purified cell population; or extra-cellular matrix (ECM) component; or an intra-cellular matrix (ICM) component that mayor may not be purified or a synthetically produced analog. Additionally, refined, purified, or synthetic analogs of polysaccharides, proteoglycans, cellulose species or other bio-mimetic molecules or derived from animal or plant sources should be considered as part of a tissue component. As discussed, the tissue component may be in particulate form. It may also act as a porogen when removed from the polyurethane matrix. In some embodiments the Tissue Component is a filler or a porogen, and thus these terms are used synonymously with regard to certain embodiments disclosed herein.

Any kind of tissue and/or tissue-derived components may be used in the present invention. In some embodiments, tissue components employed in the preparation of tissue component containing composites are obtained from tissue. A tissue component may be obtained from any vertebrate, or non-vertebrate animal or plant species. Tissue components may be of autogenous, allogenic, and/or xenogeneic origin. In certain embodiments, tissue components are autogenous, that is, tissue components are from the subject being treated. In other embodiments, tissue components are allogenic (e.g., from donors). In certain embodiments, the source of tissue may be matched to the eventual recipient of inventive composites (i.e., the donor and recipient are of the same species). For example, human tissue components are typically used in a human subject. In certain embodiments, tissue components are obtained from tissue of allogenic origin. In certain embodiments, tissue components are obtained from tissue of xenogeneic origin. Porcine and bovine tissue are types of xenogeneic tissue that can be used individually or in combination as sources for tissue components and may offer advantageous properties. Xenogenic tissue may be combined with allogenic or autogenous tissue.

In certain embodiments of the invention the tissue component is extracellular matrix sub-component or sub-components (e.g., collagen or other matrix proteins, hyaluronic acid or other polysaccharides), or synthetic analog components (e.g., carboxymethyl cellulose). In such embodiments the tissue component absorbs moisture from the wound bed, thus limiting over-expansion of the foam due to diffusion of water from the host tissue into the injected material. The tissue component also precludes both the formation of non-functional excessively large voids, as well as an undesirable pore morphology due to the excessively large pores that result from the diffusion of water or interstitial fluids from the wound bed into the reacting PUR portion of the composite. The tissue component is specifically engineered to absorb moisture from the wound bed, resulting in controlled expansion and pore morphology formation. Either during or after cure of the PUR component, the tissue component is removed from the injected material either through the process of dissolution or by cell-mediated degradation, thereby creating additional pores. Therefore in preferred embodiments the tissue component also functions as a porogen. The Tissue Component also allows for adhesive type of binding to host tissue.

In some embodiments, the tissue component may be a carbohydrate, which may also serve as a porogen. A carbohydrate may be a monosaccharide, disaccharide, or polysaccharide. The carbohydrate may be a natural or synthetic carbohydrate. In some embodiments, the carbohydrate is a biocompatible, biodegradable carbohydrate. In certain embodiments, the carbohydrate is a polysaccharide. Exemplary polysaccharides include cellulose, starch, HA, CMC, amylose, dextran, poly(dextrose), glycogen, etc. In certain embodiments, a polysaccharide is dextran. Very high molecular weight dextran has been found particularly useful as a porogen. For example, the molecular weight of the dextran may range from about 500,000 glmol to about 10,000,000 glmol, preferably from about 1,000,000 glmol to about 3,000,000 glmol. In certain embodiments, the dextran has a molecular weight of approximately 2,000,000 glmol. Dextrans with a molecular weight higher than 10,000,000 glmol may also be used as porogens. Dextran may be used in any form (e.g., particles, granules, fibers, elongated fibers) as a porogen. In certain embodiments, fibers or elongated fibers of dextran are used as a porogen in inventive composites. Fibers of dextran may be formed using any known method including extrusion and precipitation. Fibers may be prepared by precipitation by adding an aqueous solution of dextran (e.g., 5-25% dextran) to a less polar solvent such as a 90-100% alcohol (e.g., ethanol) solution. The dextran precipitates out in fibers that are particularly useful as porogens in the inventive composite. Once the composite with dextran as a tissue component porogen is used, the dextran dissolves away very quickly. Within approximately 24 hours, substantially all of dextran is out of composites leaving behind pores in the composite. An advantage of using dextran in a composite is that dextran exhibits a hemostatic property in extravascular space. Therefore, dextran in a composite can decrease bleeding at or near the site of use.

Tissue components can be formed by any process known to break down tissue into small pieces or subcomponents. Exemplary processes for forming such components include tissue graft harvesting, milling, cell purification, or ECM or ICM purification or synthesis. Exemplary particulate shapes include spheroidal, plates, shards, fibers, cuboidal, sheets, rods, oval, strings, elongated components, wedges, discs, rectangular, polyhedral, etc.

As for irregularly shaped tissue components, recited dimension ranges may represent the length of the greatest or smallest dimension of the particle. As examples, tissue components can be pin shaped, with tapered ends having an average diameter of from about 100 microns to about 500 microns. As will be appreciated by one of skill in the art, for injectable composites, the maximum particle size will depend in part on the size of the cannula or needle through which the material will be delivered.

In some embodiments, size distribution of tissue components utilized in accordance with the present inventions with respect to a mean value or a median value may be plus or minus, e.g., about 10% or less of the mean value, about 20% or less of the mean value, about 30% or less of the mean value, about 40% or less of the mean value, about 50% or less of the mean value, about 60% or less of the mean value, about 70% or less of the mean value, about 80% or less of the mean value, or about 90% or less of the mean value.

In some embodiments, particulate tissue components may have a median or mean diameter or a median or mean length of about 1200 microns, 1100 microns, 1000 microns, 900 microns, 800 microns, 700 microns, 600 microns, 500 microns, 400 microns, 300 microns, 200 microns, 100 microns, etc. In some embodiments, diameters of tissue components are within a range between any of such sizes. Furthermore, median or mean diameters or lengths of tissue components have a range from approximately 1 micron to approximately 5000 microns. In some embodiments, about 70, about 80 or about 90 percent of tissue components possess a median or mean diameter or a median or mean length within a range of any of such sizes.

For tissue components that are fibers or other elongated components, in some embodiments, at least about 90 percent of the components possess a median or mean length in their greatest dimension in a range from approximately 100 microns to approximately 1000 microns. Components may possess a median or mean length to median or mean thickness ratio from at least about 5:1 up to about 500:1, for example, from at least about 50:1 up to about 500:1, or from about 50:1 up to about 100:1; and a median or mean length to median or mean width ratio of from about 10:1 to about 200:1 and, for example, from about 50:1 to about 100:1. In certain embodiments, tissue components may short fibers having a cross section of about 300 microns to about 100 microns and a length of about 0.1 mm to about 1 mm.

Processing of tissue components to provide sub-components may be adjusted to optimize for the desired size and/or distribution of tissue components or components. The properties of resulting inventive composites (e.g., mechanical properties or degradation profile) may also be engineered by adjusting weight percent, shapes, sizes, distribution, etc. of tissue components or components or other components. For example, an inventive composite may be made more viscous and load bearing by including a higher percentage of components.

The surfaces of particulate tissue components utilized in accordance with the present invention may be optionally treated to enhance their interaction with polyurethanes and/or to confer some properties to particle surface. While some particulate tissue components will interact readily with monomers and be covalently linked to polyurethane matrices, it may be desirable to modify the surface of tissue components to facilitate their incorporation into polymers that do not bond well to tissue, such as poly(lactides). Surface modification may provide a chemical substance that is strongly bonded to the surface of tissue, e.g., covalently bonded to the surface. Particulate tissue components may, alternatively or additionally, be coated with a material to facilitate interaction with polymers of inventive composites.

Alternatively or additionally, biologically active compounds such as a biomolecule, a small molecule, or a bioactive agent may be attached to tissue components through a linker. For example, mercaptosilanes will react with sulfur atoms in proteins to attach them to tissue components. Aminated, hydroxylated, and carboxylated silanes will react with a wide variety functional groups. Of course, the linker may be optimized for the compound being attached to tissue components.

Biologically active molecules can modify non-mechanical properties of inventive composites as they degrade. For example, immobilization of a drug on tissue components allows it to be gradually released at an implant site as the composite degrades. Antiinflammatory agents embedded within inventive composites will control inflammatory response long after an initial response to injection of the composites. For example, if a piece of the composite fractures several weeks after injection, immobilized compounds will reduce the intensity of any inflammatory response, and the composite will continue to degrade through hydrolytic or physiological processes. In some embodiments, compounds may also be immobilized on the tissue components that are designed to elicit a particular metabolic response or to attract cells to injection sites.

Some biomolecules, small molecules, and bioactive agents may also be incorporated into PUR matrices used in embodiments of the present invention. For example, many amino acids have reactive side chains. The phenol group on tyrosine has been exploited to form polycarbonates, polyarylates, and polyiminocarbonates (see Pulapura, et al., Biopolymers, 1992, 32: 411-417; and Hooper, et al., J Bioactive and Compatible Polymers, 1995, 10:327-340, the entire contents of both of which are incorporated herein by reference). Amino acids such as lysine, arginine, hydroxylysine, proline, and hydroxyproline also have reactive groups and are essentially tri-functional. Amino acids such as valine, which has an isopropyl side chain, are still difunctional. Such amino acids may be attached to the silane and still leave one or two active groups available for incorporation into a polymer.

Non-biologically active materials may also be attached to tissue components. For example, radiopaque (e.g., barium sulfate), luminescent (e.g., quantum dots), or magnetically active components (e.g., iron oxide) may be attached to tissue components using the techniques described above. Mineralized tissue components are an inherently radiopaque component of some embodiments of present inventions, whereas demineralized tissue components, another optional component of inventive composites, are not radiopaque. To enhance radiopacity of inventive composites, mineralized tissue components can be used. Another way to render radiopaque the polymers utilized in accordance with the present invention is to chemically modify them such that a halogen (e.g., iodine) is chemically incorporated into the polyurethane matrices, as in U.S. Patent Publication No. 2006-0034769, whose content is incorporated herein by reference.

If a material, for example, an alloplastic or tissue transplant atom or cluster, cannot be produced as a silane or other group that reacts with tissue components, then a chelating agent may be immobilized on tissue particle surface and allowed to form a chelate with the atom or cluster. As tissue components and polymers used in the present invention are resorbed, these non-biodegradable materials may be removed from tissue sites by natural metabolic processes, allowing degradation of the polymers and resorption of the tissue components to be tracked using standard medical diagnostic techniques.

Collagen fibers exposed by demineralization are typically relatively inert but have some exposed amino acid residues that can participate in reactions. Collagen may be rendered more reactive by fraying triple helical structures of the collagen to increase exposed surface area and number of exposed amino acid residues. This not only increases surface area of tissue components available for chemical reactions but also for their mechanical interactions with polymers as well. Rinsing partially demineralized tissue components in an alkaline solution will fray collagen fibrils. For example, tissue components may be suspended in water at a pH of about 10 for about 8 hours, after which the solution is neutralized. One skilled in the art will recognize that this time period may be increased or decreased to adjust the extent of fraying. Agitation, for example, in an ultrasonic bath, may reduce the processing time. Alternatively or additionally, tissue components may be sonicated with water, surfactant, alcohol, or some combination of these.

In some embodiments, collagen fibers at tissue component particle surface may be cross-linked. A variety of cross-linking techniques suitable for medical applications are well known in the art (see, for example, U.S. Pat. No. 6,123,781, the contents of which are incorporated herein by reference). For example, compounds like 1-ethyl-3-(3-dimethylaminopropyl) carbodiimide hydrochloride, either alone or in combination with Nhydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, may also be used to cross-link collagen chains (see Simmons, et al, Biotechnol. Appl. Biochem., 1993, 17:23-29; peT Publication WO98119718, the contents of both of which are incorporated herein by reference). Alternatively or additionally, hydroxymethyl phosphine groups on collagen may be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethy lene diisocyanate, and sugars may also be used to cross-link collagens. Tissue components are then washed to remove all leachable traces of materials. In other embodiments, enzymatic cross-linking agents may be used. Additional cross-linking methods include chemical reaction, irradiation, application of heat, dehydrothermal treatment, enzymatic treatment, etc. One skilled in the art will easily be able to determine the optimal concentrations of cross-linking agents and incubation times for the desired degree of cross-linking.

Both frayed and unfrayed collagen fibers may be derivatized with monomer, pre-polymer, oligomer, polymer, initiator, and/or biologically active or inactive compounds, including but not limited to biomolecules, bioactive agents, small molecules, inorganic materials, minerals, through reactive amino acids on the collagen fiber such as lysine, arginine, hydroxy lysine, proline, and hydroxyproline. Monomers that link via step polymerization may react with these amino acids via the same reactions through which they polymerize. Vinyl monomers and other monomers that polymerize by chain polymerization may react with these amino acids via their reactive pendant groups, leaving the vinyl group free to polymerize. Alternatively, or in addition, tissue components may be treated to induce calcium phosphate deposition and crystal formation on exposed collagen fibers. Calcium ions may be chelated by chemical moieties of the collagen fibers, and/or calcium ions may bind to the surface of the collagen fibers. James et al., Biomaterials 20:2203-2313, 1999; incorporated herein by reference. The calcium ions bound to the collagen provides a biocompatible surface, which allows for the attachment of cells as well as crystal growth. The polymer will interact with these fibers, increasing interfacial area and improving the wet strength of the composite.

In some embodiments, the surface treatments described above or treatments such as etching may be used to increase the surface area or surface roughness of particulate tissue components. Such treatments increase the interfacial strength of the particle/polymer interface by increasing the surface area of the interface and/or the mechanical interlocking of tissue components and polyurethane. Such surface treatments may also be employed to round the shape or smooth the edges of tissue components to facilitate delivery of the inventive composite. Such treatment is particularly useful for injectable composites.

In some embodiments, surface treatments of tissue components are optimized to enhance covalent attractions between tissue components and polyurethanes. In some embodiments, the surface treatment may be designed to enhance non-covalent interactions between tissue particle and polyurethane matrix. Exemplary non-covalent interactions include electrostatic interactions, hydrogen bonding, pi-bond interactions, hydrophobic interactions, van der Waals interactions, and mechanical interlocking. For example, if a protein or a polysaccharide is immobilized on tissue particle, the chains of polymer matrix will become physically entangled with long chains of the biological macromolecules when they are combined. Charged phosphate sites on the surface of tissue components, produced by washing the tissue components in basic solution, will interact with the amino groups present in many biocompatible polymers, especially those based on amino acids. The pi-orbitals on aromatic groups immobilized on a tissue particle will interact with double bonds and aromatic groups of the polymer.

In some embodiments, a tissue component may be employed in combination with other materials. For example, inorganic materials such as those described, for example, in U.S. patent application Ser. Nos. 10/735,135; 10/681,651; and 10/639,912; (incorporated herein by reference) may be combined with proteins such as bovine serum albumin (BSA), collagen, or other extracellular matrix ECM or ICM components to form a composite. In some embodiments, the inventive compositions and/or composites may include a tissue component that is a polysaccharide (e.g., carboxymethylcellulose (CMC) and hyaluronic acid (HA). In certain embodiments, when composites used in wound healing, solid fillers can help absorb excess moisture in the wounds from blood and serum and allow for proper foaming. For example, see Patent Application No. PCT/US10/32327, incorporated herein by reference.

Polymer Component

Synthetic polymers can be designed with properties targeted for a given clinical application. According to the present invention, PUR are a useful class of biomaterials due to the fact that they can be injectable or moldable as a reactive liquid that subsequently cures to form a porous composite. These materials also have tunable degradation rates, which are shown to be highly dependent on the choice of polyol and isocyanate components (Hafeman et al., Pharmaceutical Research 2008; 25(10):2387-99; Storey et al., J Poly Sci Pt A: Poly Chem 1994; 32:2345-63; Skarja et al., J App Poly Sci 2000; 75:1522-34). Polyurethanes have tunable mechanical properties, which can also be enhanced with the addition of tissue components or subcomponents and/or other components (Adhikari et al., Biomaterials 2008; 29:3762-70; Goma et al., J Biomed Mater Res Pt A 2003; 67A(3):813-27) and exhibit elastomeric rather than brittle mechanical properties.

U.S. Pat. No. 6,306,177, discloses a method for repairing a tissue site using PUR, the content of which is incorporated by reference.

It is to be understood that by “a two-component composition” it means a composition comprising two essential types of polymer components. In some embodiments, such a composition may additionally comprise one or more other optional components.

In some embodiments, polyurethane is a polymer that has been rendered formable through combination of two liquid components (i.e., a polyisocyanate prepolymer and a polyol). In some embodiments, a polyisocyanate prepolymer or a polyol may be a molecule with two or three isocyanate or hydroxyl groups respectively. In some embodiments, a polyisocyanate prepolymer or a polyol may have at least four isocyanate or hydroxyl groups respectively.

Synthesis of porous polyurethane results from a balance of two simultaneous reactions. Reactions, in some embodiments, are illustrated below in Scheme 1. One is a gelling reaction, where an isocyanates and a polyester polyol react to form urethane bonds. The one is a blowing reaction. An isocyanate can react with water to form carbon dioxide gas, which acts as a lowing agent to form pores of polyurethane foam. The relative rates of these reactions determine the scaffold morphology, working time, and setting time.

Exemplary gelling and blowing reactions in forming of polyurethane are shown in Scheme 1 below, where R₁, R₂ and R₃, for example, can be oligomers of caprolactone, lactide and glycolide respectively.

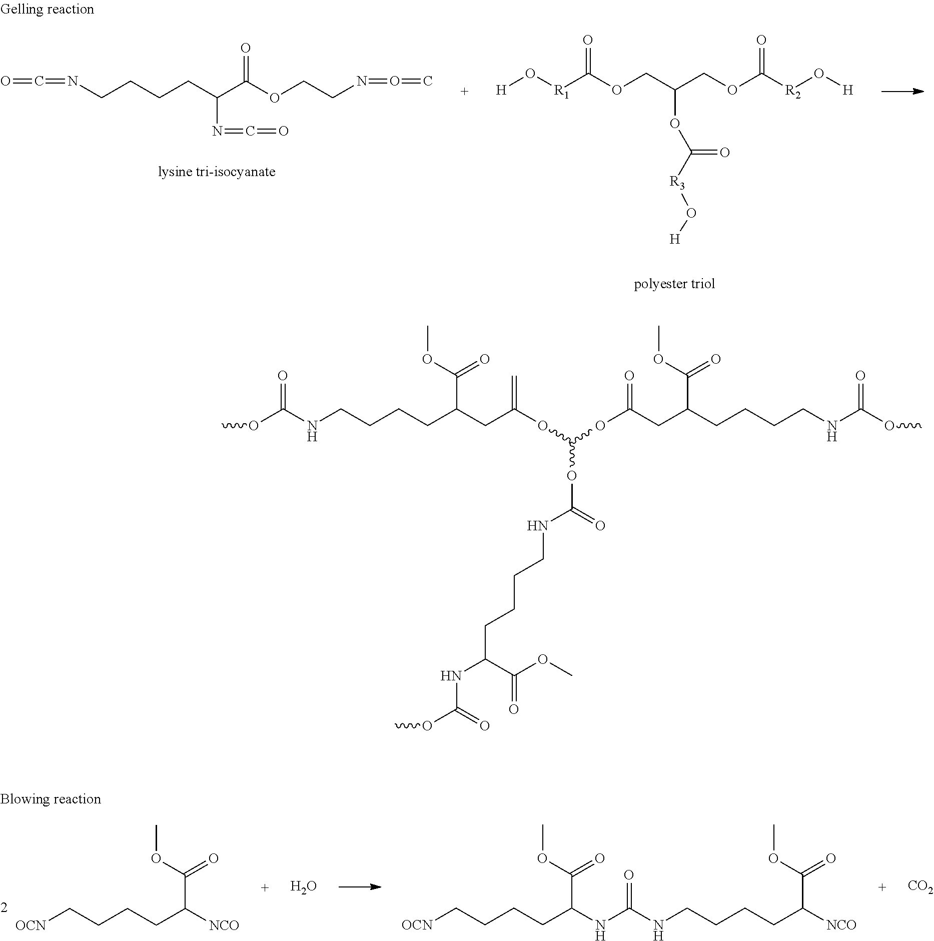

Biodegradable polyurethane scaffolds synthesized from aliphatic polyisocyanates may degrade into non-toxic compounds and support cell attachment and proliferation in vitro. A variety of polyurethane polymers suitable for use in the present invention are known in the art, many of which are listed in commonly owned applications: U.S. Ser. No. 10/759,904 filed on Jan. 16, 2004, entitled “Biodegradable polyurethanes” and use thereof and published under No. 2005/0013793; U.S. Ser. No. 11/667,090 filed on Nov. 5, 2005, entitled “Degradable polyurethane foams” and published under No. 2007/0299151; U.S. Ser. No. 12/298,158 filed on Apr. 24, 2006, entitled “Biodegradable polyurethanes” and published under No. 2009/0221784; all of which are incorporated herein by reference. Polyurethanes described in U.S. Ser. No. 11/336,127 filed on Jan. 19, 2006 and published under No. 2006/0216323, which is entitled “Polyurethanes for Osteoimplants” and incorporated herein by reference, may be used in some embodiments of the present invention. PUR foams may be prepared by contacting an isocyanate-terminated prepolymer (component 1, e.g, polyisocyanate prepolymer) with a hardener (component 2) that includes at least a polyol (e.g., a polyester polyol) and water, a catalyst and optionally, a stabilizer, a porogen, pore opener, PEG, etc. In some embodiments, multiple polyurethanes (e.g., different structures, difference molecular weights) may be used in a composite/composition of the present invention. In some embodiments, other biocompatible and/or biodegradable polymers may be used with polyurethanes in accordance with the present invention. In some embodiments, biocompatible co-polymers and/or polymer blends of any combination thereof may be exploited.

Polyurethanes used in accordance with the present invention can be adjusted to produce polymers having various physiochemical properties and morphologies including, for example, flexible foams, rigid foams, elastomers, coatings, adhesives, and sealants. The properties of polyurethanes are controlled by choice of the raw materials and their relative concentrations. For example, thermoplastic elastomers are characterized by a low degree of cross-linking and are typically segmented polymers, consisting of alternating hard (diisocyanates and chain extenders) and soft (polyols) segments. Thermoplastic elastomers are formed from the reaction of diisocyanates with long-chain diols and short-chain diol or diamine chain extenders. In some embodiments, pores in tissue/polyurethanes composites in the present invention are interconnected and have a diameter ranging from approximately 50 to approximately 1000 microns.

Prepolymer.

PUR prepolymers may be prepared by contacting a polyol with an excess (typically a large excess) of a polyisocyanate. The resulting prepolymer intermediate includes an adduct of polyisocyanates and polyols solubilized in an excess of polyisocyanates. Prepolymer can, in some embodiments, be formed by using an approximately stoichiometric amount of polyisocyanates in forming a prepolymer and subsequently adding additional polyisocyanates. The prepolymer therefore exhibits both low viscosity, which facilitates processing, and improved miscibility as a result of the polyisocyanate-polyol adduct. Polyurethane networks can, for example, then be prepared by reactive liquid molding, wherein the prepolymer is contacted with a polyester polyol to form a reactive liquid mixture (i.e., a two-component composition) which is then cast into a mold and cured.

Polyisocyanates or multi-isocyanate compounds for use in the present invention include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate (LTI), hexamethylene diisocyanate, isophorone diisocyanate (1PDI), 4,4′-dicyclohexylmethane diisocyanate (H12MDI), cyclohexyl diisocyanate, 2,2,4-(2,2,4)-trimethylhexamethylene diisocyanate (TMOI), dimers prepared form aliphatic polyisocyanates, trimers prepared from aliphatic polyisocyanates and/or mixtures thereof. In some embodiments, hexamethylene diisocyanate (HOI) trimer sold as Desmodur N3300A may be a polyisocyanate utilized in the present invention. In some embodiments the polyisocyanates include lysine methyl ester diisocyanate, lysine triisocyanate, 1,4-diisocyanatobutane, or hexamethylene diisocyanate. In some embodiments, polyisocyanates used in the present invention includes approximately 10 to 55% NCO by weight (wt % NCO=100*(42IMw)). In some embodiments, polyisocyanates include approximately 15 to 50% NCO.

Poly isocyanate prepolymers provide an additional degree of control over the structure of biodegradable PUR. Prepared by reacting polyols with isocyanates, NCO-terminated prepolymers are oligomeric intermediates with isocyanate functionality. To increase reaction rates, urethane catalysts (e.g., tertiary amines) and/or elevated temperatures (60-90 DC) may be used (see, Guelcher, Tissue Engineering: Part B, 14(1)2008, pp. 3-17). Prepolymers (e.g., LTI-PEG prepolymers) can also have the advantage of being no cytotoxic or being less cytotoxic than monomeric polyisocyanate (e.g., LTI) in vivo.¹³

Polyols used to react with polyisocyanates in preparation of NCO-terminated prepolymers may refer to molecules having at least two functional groups to react with isocyanate groups. In some embodiments, polyols have a molecular weight of no more than 1000 g/mol. In some embodiments, polyols have a range of molecular weight between about 100 g/mol to about 500 g/mol. In some embodiments, polyols have a range of molecular weight between about 200 g/mol to about 400 g/mol. In certain embodiments, polyols (e.g., PEG) have a molecular weight of about 200 g/mol. Exemplary polyols include, but are not limited to, PEG, glycerol, pentaerythritol, dipentaerythritol, tripentaerythritol, 1,2,4-butanetriol, trimethylolpropane, 1,2,3-trihydroxyhexane, myo-inositol, ascorbic acid, a saccharide, or sugar alcohols (e.g., mannitol, xylitol, sorbitol etc.). In some embodiments, polyols may comprise multiple chemical entities having reactive hydrogen functional groups (e.g., hydroxy groups, primary amine groups and/or secondary amine groups) to react with the isocyanate functionality of polyisocyanates.

In some embodiments, polyisocyanate prepolymers are resorbable. Zhang and coworkers synthesized biodegradable lysine diisocyanate ethyl ester (LOI)/glucose polyurethane foams proposed for tissue engineering applications. In those studies, NCO-terminated prepolymers were prepared from LDI and glucose. The prepolymers were chain extended with water to yield biocompatible foams which supported the growth of rabbit tissue marrow stromal cells in vitro and were non-immunogenic in vivo. (see Zhang, et al., Biomaterials 21: 1247-1258 (2000), and Zhang, et al., Tiss. Eng., 8(5): 771-785 (2002), both of which are incorporated herein by reference).

In some embodiments, prepared polyisocyanate prepolymer can be a flowable liquid at processing conditions. In certain embodiments, the processing temperature is no greater than 60° C. In some embodiments, the processing temperature is ambient temperature (25° C.).

In some embodiments the ratio of polyisocyanate to polyol can be adjusted to modify different characteristics of the prepolymer, including its reactivity, viscosity, or the like. In this regard, some embodiments of prepolymers comprise a 2:1 molar ratio of polyisocyanate to polyol. In other embodiments the molar ratio of polyisocyanate to polyol is about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, or about 3.0:1.

In this regard, the viscosity of the prepolymer can also vary depending on different factors. In some embodiments the viscosity of the prepolymer will vary depending on the molar ratio of polyisocyanate to polyol that is used. The viscosity can be tuned so that the composite has desirable workable characteristics (e.g., injectable, putty, etc.), among other things. In some embodiments the viscosity of the prepolymer can be about 10,000 cSt, about 11,000 cSt, about 12,000 cSt, about 13,000 cSt, about 14,000 cSt, about 15,000 cSt, about 16,000 cSt, about 17,000 cSt, about 18,000 cSt, about 19,000 cSt, about 20,000 cSt, about 21,000 cSt, about 22,000 cSt, about 23,000 cSt, about 24,000 cSt, about 25,000 cSt, about 26,000 cSt, about 27,000 cSt, about 28,000 cSt, about 29,000 cSt, or about 30,000 cSt.

Polyols.

Polyols, which are biocompatible, utilized in accordance with the present invention can be amine- and/or hydroxyl-terminated compounds and include, but are not limited to, polyether polyols (such as polyethylene glycol (PEG) or polyethylene oxide (PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). In some embodiments, polyols may be (1) molecules having multiple hydroxyl or amine functionality, such as glucose, polysaccharides, and castor oil; and (2) molecules (such as fatty acids, triglycerides, and phospholipids) that have been hydroxylated by known chemical synthesis techniques to yield polyols.

Polyols used in the present invention may be polyester polyols. In some embodiments, polyester polyols may include polyalkylene glycol esters or polyesters prepared from cyclic esters. In some embodiments, polyester polyols may include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. In some embodiments, polyester polyols can include, polyesters prepared from caprolactone, glycolide, D, L-Iactide, mixtures thereof, and/or copolymers thereof. In some embodiments, polyester polyols can, for example, include polyesters prepared from castor-oil. When polyurethanes degrade, their degradation products may be the polyols from which they were prepared from.

In some embodiments, polyester polyols can be miscible with prepared prepolymers used in reactive liquid mixtures (i.e., two-component composition) of the present invention. In some embodiments, surfactants or other additives may be included in the reactive liquid mixtures to help homogenous mixing.

The glass transition temperature (Tg) of polyester polyols used in the reactive liquids to form polyurethanes can be less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

Molecular weight of polyester polyols used in the reactive liquids to form polyurethanes can, for example, be adjusted to control the mechanical properties of polyurethanes utilized in accordance with the present invention. In that regard, using polyester polyols of higher molecular weight results in greater compliance or elasticity. In some embodiments, polyester polyols used in the reactive liquids may have a molecular weight less than approximately 3000 Da. In certain embodiments, the molecular weight may be in the range of approximately 200 to 2500 Da or 300 to 2000 Da. In some embodiments, the molecular weight may be approximately in the range of approximately 450 to 1800 Da or 450 to 1200 Da. In some embodiments, a polyester polyol comprise poly(caprolactone-colactide-co-glycolide), which has a molecular weight in a range of 200 Da to 2500 Da, or 300 Da to 2000 Da.

In some embodiments, polyols may include multiply types of polyols with different structures, molecular weight, properties, etc.

Additional Components.

In accordance with the present invention, two component compositions (i.e., polyprepolymers and polyols) to form porous composites may be used with other agents and/or catalysts. Zhang et at. have found that water may be an adequate blowing agent for a lysine diisocyanatelPEG/glycerol polyurethane (see Zhang, et al., Tissue Eng. 2003 (6):1143-57) and may also be used to form porous structures in polyurethanes. Other blowing agents include dry ice or other agents that release carbon dioxide or other gases into the composite. Alternatively, or in addition, porogens (see detail discussion below) such as salts may be mixed in with reagents and then dissolved after polymerization to leave behind small voids.

Two-component compositions and/or the prepared composites used in the present invention may include one or more additional components. In some embodiments, inventive compositions and/or composites may includes, water, a catalyst (e.g., gelling catalyst, blowing catalyst, etc.), a stabilizer, a plasticizer, a porogen, a chain extender (for making of polyurethanes), a pore opener (such as calcium stearate, to control pore morphology), a wetting or lubricating agent, etc. (See, U.S. Ser. No. 10/759,904 published under No. 2005/0013793, and U.S. Ser. No. 11/625,119 published under No. 2007/0191963; both of which are incorporated herein by reference).

Water. Water may be a blowing agent to generate porous polyurethane-based composites. Porosity of tissue/polymer composites increased with increasing water content, and biodegradation rate accelerated with decreasing polyester half-life, thereby yielding a family of materials with tunable properties that are useful in the present invention. See, Guelcher et al., Tissue Engineering, 13(9), 2007, pp 2321-2333, which is incorporated by reference. In some embodiments, an amount of water is about 0, 0.5, 1, 1.5, 2, 3, 4, 5, 6, 7, 8, 9, 10 parts per hundred parts (pphp) polyol. In some embodiments, water has an approximate range of any of such amounts.

Catalyst.

In some embodiments, at least one catalyst is added to form reactive liquid mixture (i.e., two-component compositions). A catalyst, for example, can be non-toxic (in a concentration that may remain in the polymer). A catalyst can, for example, be present in two-component compositions in a concentration in the range of approximately 0.375 to 5 parts per hundred parts polyol (pphp) and, for example, in the range of approximately 0.5 to 2, or 2 to 3 pphp. A catalyst can, for example, be an amine compound. In some embodiments, catalyst may be an organometallic compound or a tertiary amine compound, such as TEGOAMIN33, for example. In some embodiments the catalyst may be stannous octoate (an organobismuth compound), triethylene diamine, bis(dimethylaminoethyl)ether, dimethylethanolamine, dibutyltin dilaurate, and Coscat organometallic catalysts manufactured by Vertullus (a bismuth based catalyst), or any combination thereof.

Stabilizer.

In some embodiments, a stabilizer is nontoxic (in a concentration remaining in the polyurethane foam) and can include a non-ionic surfactant, an anionic surfactant or combinations thereof. For example, a stabilizer can be a polyethersiloxane, a salt of a fatty sulfonic acid or a salt of a fatty acid. In certain embodiments, a stabilizer is a polyethersiloxane, and the concentration of polyethersiloxane in a reactive liquid mixture can, for example, be in the range of approximately 0.25 to 4 pphp. In some embodiments, polyethersiloxane stabilizer are hydrolyzable.

In some embodiments, the stabilizer can be a salt of a fatty sulfonic acid. Concentration of a salt of the fatty sulfonic acid in a reactive liquid mixture can be in the range of approximately 0.5 to 5 parts per hundred polyol. Examples of suitable stabilizers include a sulfated castor oil or sodium ricinoleicsulfonate.

Stabilizers can be added to a reactive liquid mixture of the present invention to, for example, disperse prepolymers, polyols and other additional components, stabilize the rising carbon dioxide bubbles, and/or control pore sizes of inventive composites. Although there has been a great deal of study of stabilizers, the operation of stabilizers during foaming is not completely understood. Without limitation to any mechanism of operation, it is believed that stabilizers preserve the thermodynamically unstable state of a polyurethane foam during the time of rising by surface forces until the foam is hardened. In that regard, foam stabilizers lower the surface tension of the mixture of starting materials and operate as emulsifiers for the system. Stabilizers, catalysts and other polyurethane reaction components are discussed, for example, in Oertel, Gunter, ed., Polyurethane Handbook, Hanser Gardner Publications, Inc. Cincinnati, Ohio, 99-108 (1994). A specific effect of stabilizers is believed to be the formation of surfactant monolayers at the interface of higher viscosity of bulk phase, thereby increasing the elasticity of surface and stabilizing expanding foam bubbles.

Chain Extender.

To prepare high-molecular-weight polymers, prepolymers are chain extended by adding a short-chain (e.g., <500 g/mol) polyamine or polyol. In certain embodiments, water may act as a chain extender. In some embodiments, addition of chain extenders with a functionality of two (e.g., diols and diamines) yields linear alternating block copolymers.

Plasticizer.

In some embodiments, inventive compositions and/or composites include one or more plasticizers. Plasticizers are typically compounds added to polymers or plastics to soften them or make them more pliable. According to the present invention, plasticizers soften, make workable, or otherwise improve the handling properties of polymers or composites. Plasticizers also allow inventive composites to be moldable at a lower temperature, thereby avoiding heat induced tissue necrosis during implantation. Plasticizer may evaporate or otherwise diffuse out of the composite over time, thereby allowing composites to harden or set. Without being bound to any theory, plasticizer are thought to work by embedding themselves between the chains of polymers. This forces polymer chains apart and thus lowers the glass transition temperature of polymers. In general, the more plasticizer added, the more flexible the resulting polymers or composites will be.

In some embodiments, plasticizers are based on an ester of a polycarboxylic acid with linear or branched aliphatic alcohols of moderate chain length. For example, some plasticizers are adipate-based. Examples of adipate-based plasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl) phthalate (DEHP), diisononyl phthalate (DINP), bis(nbutyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid poly hydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl)trimellitate (TEHTM-MG), tri-(n-octyl,n-decyl)trimellitate (ATM), tri-(heptyl,nonyl)trimellitate (LTM), n-octyl trimellitate (OTM)), benzoates, epoxidized vegetable oils, sulfonamides (e.g., N-ethyl toluene sulfonamide (ETSA), N-(2-hydroxypropyl)benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to the composite as plasticizers. In certain particular embodiments, polymers with the same chemical structure as those used in the composite are used but with lower molecular weights to soften the overall composite. In other embodiments, different polymers with lower melting points and/or lower viscosities than those of the polymer component of the composite are used.

In some embodiments, a polymers used as plasticizer are poly(ethylene glycol) (PEG). PEG, which also may be used as a plasticizer, is typically a low molecular weight PEG such as those having an average molecular weight of 1000 to 10000 g/mol, for example, from 4000 to 8000 g/mol. In certain embodiments, as discussed here and above, PEG 4000, PEG 5000, PEG 6000, PEG 7000, PEG 8000 or combinations thereof may be used in inventive composites. For example, plasticizer (PEG) is useful in making more moldable composites that include poly(lactide), poly(D,L-lactide), poly(lactide-co-glycolide), poly(D,L-lactide-co-glycolide), or poly(caprolactone). Plasticizer may comprise 1-40% of inventive composites by weight. In some embodiments, the plasticizer is 10-30% by weight. In some embodiments, the plasticizer is approximately 10%, 15%, 20%, 25%, 30% or 40% by weight. In other embodiments, a plasticizer is not used in the composite. For example, in some polycaprolactone-containing composites, a plasticizer is not used.

In some embodiments, inert plasticizers may be used. In some embodiments, a plasticizer may not be used in the present invention.

Additional Porogens.

Porosity of inventive composites may be accomplished using any means known in the art. Exemplary methods of creating porosity in a composite include, but are not limited to, particular leaching processes, gas foaming processing, supercritical carbon dioxide processing, sintering, phase transformation, freeze-drying, cross linking, molding, porogen melting, polymerization, melt-blowing, and salt fusion (Murphy et al., Tissue Engineering 8(1):43-52, 2002; incorporated herein by reference). For a review, see Karageorgiou et al., Biomaterials 26:5474-5491, 2005; incorporated herein by reference. Porosity may be a feature of inventive composites during manufacture or before implantation, or porosity may only be available after implantation. For example, a implanted composite may include latent pores. These latent pores may arise from including porogens in the composite. In some embodiments the tissue component will function as the porogen. Some embodiments of the invention that comprise a tissue component that is a porogen can further include one or more other porogens to modify porosity.

Porogens may be any chemical compound that will reserve a space within the composite while the composite is being molded and will diffuse, dissolve, and/or degrade prior to or after implantation or injection leaving a pore in the composite. Porogens may have the property of not being appreciably changed in shape and/or size during the procedure to make the composite moldable. For example, a porogen should retain its shape during the heating of the composite to make it moldable. Therefore, a porogen does not melt upon heating of the composite to make it moldable. In certain embodiments, a porogen has a melting point greater than about 60° C., greater than about 70 DC, greater than about 80° C., greater than about 85 DC, or greater than about 90° C.

Porogens may be of any shape or size. A porogen may be spheroidal, cuboidal, rectangular, elonganted, tubular, fibrous, disc-shaped, platelet-shaped, polygonal, etc. In certain embodiments, the porogen is granular with a diameter ranging from approximately 100 microns to approximately 800 microns. In certain embodiments, a porogen is elongated, tubular, or fibrous. Such porogens provide increased connectivity of pores of inventive composite and/or also allow for a lesser percentage of the porogen in the composite.

Amount of porogens may vary in inventive composite from 1% to 80% by weight. In certain embodiments, the plasticizer makes up from about 5% to about 80% by weight of the composite. In certain embodiments, a plasticizer makes up from about 10% to about 50% by weight of the composite. Pores in inventive composites are thought to improve the cell and tissue inductivity or conductivity of the composite by providing holes for cells such as mononuclear and macrophage, fibroblasts, cells of the mesechymal lineage, stem cells, etc. Pores provide inventive composites with biological in growth capacity. Pores may also provide for easier degradation of inventive composites as tissue is formed and/or remodeled. In some embodiments, a porogen is biocompatible.

A porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the implant before or after implantation thereby providing pores for biological in-growth. Solid porogens may be crystalline or amorphous. Examples of possible solid porogens include water soluble compounds. Exemplary porogens include carbohydrates (e.g., sorbitol, dextran (poly(dextrose)), starch), salts, sugar alcohols, natural polymers, synthetic polymers, and small molecules.

Small molecules including pharmaceutical agents may also be used as porogens in the inventive composites. Examples of polymers that may be used as plasticizers include poly(vinyl pyrollidone), pullulan, poly(glycolide), poly(lactide), and poly(lactide-coglycolide). Typically low molecular weight polymers are used as porogens. In certain embodiments, a porogen is poly(vinyl pyrrolidone) or a derivative thereof. Plasticizers that are removed faster than the surrounding composite can also be considered porogens.

In some embodiments, a pore opener can be used to facilitate an interconnected, or open, pore structure. Such pore openers are preferably nontoxic. Exemplary pore openers are described, for example, in US Published application 2009-0130174 A1, which is incorporated herein by references.

For example, powdered divalent salts of stearic acid can be used, as they cause a local disruption of the pore structure during the foaming process and thereby gaps in the pore walls for an open pore structure.

Components to Deliver:

Alternatively or additionally, composites of the present invention may have one or more components to deliver when implanted, including biomolecules, small molecules, bioactive agents, etc., to promote tissue growth and regeneration, and/or to accelerate healing. Examples of materials that can be incorporated include chemotactic factors, angiogenic factors, tissue cell inducers and stimulators, including the general class of cytokines such as the TGF-J3 super family of tissue growth factors, the family of tissue morphogenic proteins, osteoinductors, and/or tissue marrow or tissue forming precursor cells, isolated using standard techniques. Sources and amounts of such materials that can be included are known to those skilled in the art.

Biologically active materials, comprising biomolecules, small molecules, and bioactive agents may also be included in inventive composites to, for example, stimulate particular metabolic functions, recruit cells, or reduce inflammation. For example, nucleic acid vectors, including plasmids and viral vectors, that will be introduced into the patient's cells and cause the production of growth factors such as tissue morphogenetic proteins may be included in a composite. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, antiinflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, tissue digester, antitumor agent, cellular attractant, fibronectin, growth hormone cellular attachment agent, immunosuppressant, nucleic acid, surface active agent, hydroxyapatite, and penetraction enhancer. Additional exemplary substances include chemotactic factors, angiogenic factors, analgesics, antibiotics, anti-inflammatory agents, tissue morphogenic proteins, and other growth factors that promote cell-directed degradation or remodeling of the polymer phase of the composite and/or development of new tissue (e.g., tissue). RNAi or other technologies may also be used to reduce the production of various factors.

In some embodiments, inventive composites include antibiotics. Antibiotics may be bacteriocidial or bacteriostatic. An anti-microbial agent may be included in composites. For example, anti-viral agents, anti-protazoal agents, anti-parasitic agents, etc. may be include in composites. Other suitable biostaticlbiocidal agents include antibiotics, povidone, sugars, and mixtures thereof. Exemplary antibiotics include, but not limit to, Amikacin, Gentamicin, Kanamycin, Neomycin, Netilmicin, Streptomycin, Tobramycin, Paromomycin, Geldanamycin, Herbimycin, Loravabef, etc. (See, The Merck Manual of Medical Information Home Edition, 1999).

Inventive composites may also be seeded with cells. In some embodiments, a patient's own cells are obtained and used in inventive composites. Certain types of cells (e.g., osteoblasts, fibroblasts, stem cells, cells of the osteoblast lineage, etc.) may be selected for use in the composite. Cells may be harvested from marrow, blood, fat, bone, muscle, connective tissue, skin, or other tissues or organs. In some embodiments, a patient's own cells may be harvested, optionally selected, expanded, and used in the inventive composite. In other embodiments, a patient's cells may be harvested, selected without expansion, and used in the inventive composite. Alternatively, exogenous cells may be employed. Exemplary cells for use with the invention include mesenchymal stem cells and connective tissue cells, including osteoblasts, osteoclasts, fibroblasts, preosteoblasts, and partially differentiated cells of the osteoblast lineage. Cells may be genetically engineered. For example, cells may be engineered to produce a tissue morphogenic protein.

In some embodiments, inventive composites may include a composite material comprising a component to deliver. For example, a composite material can be a biomolecule (e.g., a protein) encapsulated in a polymeric microsphere or nanocomponents.

In some embodiments, inventive composites may include a composite material comprising a component to deliver locally for oncologic or chronic disease management. For example, a composite materials can be a biomolecule (e.g., a protein) encapsulated in a polymeric microsphere or nanocomponents. In certain embodiments, anti-Her2 and antiVGEF (Avastin® (bevacizumab) Herceptin® (Trastuzumab)) (Genentech, South San Francisco, Calif.) or similar bio therapeutic agents may be encapsulated in PLGA microspheres or nanoparticle spheres and embedded in the injectable polyurethane composite used in accordance with the present invention. In a patient with local or metastatic disease with positive receptor profile the tumor may be infiltrated or removed and via a minimally invasive approach fill the tumor site/tissue void with the composite of the invention. Tunable sustained release of can be achieved due to the diffusional barriers presented by both the PLGA microsphere or other Nan particulate micro spheres and polyurethane of the inventive composite.

To enhance biodegradation in vivo, composites of the present invention can also include different enzymes. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to regulate implant duration.

In some embodiments the components to deliver are not be covalently bonded to a component of the composite. In some embodiments, components can be selectively distributed on or near the surface of inventive composites using the layering techniques described above. While surface of inventive composite will be mixed somewhat as the composite is manipulated in implant site, thickness of the surface layer will ensure that at least a portion of the surface layer of the composite remains at surface of the implant. Alternatively or in addition, biologically active components may be covalently linked to the tissue components or components before combination with the polymer. As discussed above, for example, silane coupling agents having amine, carboxyl, hydroxyl, or mercapto groups may be attached to the tissue components through the silane and then to reactive groups on a biomolecule, small molecule, or bioactive agent.

Preparation of Composite

In general, inventive composites are prepared by combining components, polymers and optionally any additional components. To form inventive composites, components as discussed herein may be combined with a reactive liquid (i.e., a two component composition) thereby forming a naturally injectable or moldable composite or a composite that can be made injectable or moldable. Alternatively, components may be combined with polyisocyanate prepolymers or polyols first and then combined with other components.

In some embodiments, components may be combined first with a hardener that includes polyols, water, catalysts and optionally a solvent, a diluent, a stabilizer, a porogen, a pore opener, a plasticizer, etc., and then combined with a polyisocyanate prepolymer. In some embodiments, a hardener (e.g., a polyol, water and a catalyst) may be mixed with a prepolymer, followed by addition of components. In some embodiments, in order to enhance storage stability of two-component compositions, the two (liquid) component process may be modified to an alternative three (liquid)-component process wherein a catalyst and water may be dissolved in a solution separating from reactive polyols. For example, polyester polyols may be first mixed with a solution of a catalyst and water, followed by addition of tissue components or components, and finally addition of NCO-terminated prepolymers.

In some embodiments, additional components or components to be delivered may be combined with a reactive liquid prior to injection. In some embodiments, they may be combined with one of polymer precursors (i.e., prepolymers and polyols) prior to mixing the precursors in forming of a reactive liquid/paste.

Porous composites can be prepared by incorporating a small amount (e.g., <5 wt %) of water which reacts with prepolymers to form carbon dioxide, a biocompatible blowing agent. Resulting reactive liquid/paste may be injectable through a 12-ga syringe needle into molds or targeted site to set in situ. In some embodiments, gel time is great than 3 min, 4 min, 5 min, 6 min, 7 min, or 8 min. In some embodiments, cure time is less than 20 min, 18 min, 16 min, 14 min, 12 min, or 10 min.

In some embodiments, catalysts can be used to assist forming porous composites. In general, the more blowing catalyst used, the high porosity of inventive composites may be achieved.

Polymers and components may be combined by any method known to those skilled in the art. For example, a homogenous mixture of polymers and/or polymer precursors (e.g., prepolymers, polyols, etc.) and components may be pressed together at ambient or elevated temperatures. At elevated temperatures, a process may also be accomplished without pressure. In some embodiments, polymers or precursors are not held at a temperature of greater than approximately 60° C. for a significant time during mixing to prevent thermal damage to any biological component (e.g., growth factors or cells) of a composite. In some embodiments, temperature is not a concern because components and polymer precursors used in the present invention have a low reaction exotherm.

Alternatively or in addition, components may be mixed or folded into a polymer softened by heat or a solvent. Alternatively, a moldable polymer may be formed into a sheet that is then covered with a layer of components. Components may then be forced into the polymer sheet using pressure. In another embodiment, components are individually coated with polymers or polymer precursors, for example, using a tumbler, spray coater, or a fluidized bed, before being mixed with a larger quantity of polymer. This facilitates even coating of the components and improves integration of the components and polymer component of the composite.

After combination with components, polymers may be further modified by further cross-linking or polymerization to form a composite in which the polymer is covalently linked to the components. In some embodiments, composition hardens in a solvent-free condition. In some embodiments, compositions are a polymer/solvent mixture that hardens when a solvent is removed (e.g., when a solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, a solvent is a biological fluid such as blood, plasma, serum, marrow, etc. In certain embodiments, an inventive composite is heated above the melting or glass transition temperature of one or more of its components and becomes set after implantation as it cools. In certain embodiments, an inventive composite is set by exposing a composite to a heat source, or irradiating it with microwaves, IR rays, or UV light. Components may also be mixed with a polymer that is sufficiently pliable to combine with the components but that may require further treatment, for example, combination with a solvent or heating, to become a injectable or moldable composition. For example, a composition may be combined and injection molded, injected, extruded, laminated, sheet formed, foamed, or processed using other techniques known to those skilled in the art. In some embodiments, reaction injection molding methods, in which polymer precursors (e.g., polyisocyanate prepolymer, a polyol) are separately charged into a mold under precisely defined conditions, may be employed. For example, tissue components or components may be added to a precursor, or it may be separately charged into a mold and precursor materials added afterwards. Careful control of relative amounts of various components and reaction conditions may be desired to limit the amount of unreacted material in a composite. Post-cure processes known to those skilled in the art may also be employed. A partially polymerized polyurethane precursor may be more completely polymerized or cross-linked after combination with hydroxylated or aminated materials or included materials (e.g., a particulate, any components to deliver, etc.).

In some embodiments, an inventive composite is produced with a injectable composition and then set in situ. For example, cross-link density of a low molecular weight polymer may be increased by exposing it to electromagnetic radiation (e.g., UV light) or an alternative energy source. Alternatively or additionally, a photoactive cross-linking agent, chemical cross-linking agent, additional monomer, or combinations thereof may be mixed into inventive composites. Exposure to UV light after a composition is injected into an implant site will increase one or both of molecular weight and cross-link density, stiffening polymers (i.e., polyurethanes) and thereby a composite. Polymer components of inventive composites used in the present invention may be softened by a solvent, e.g., ethanol. If a biocompatible solvent is used, polyurethanes may be hardened in situ. In some embodiments, as a composite sets, solvent leaving the composite is released into surrounding tissue without causing undesirable side effects such as irritation or an inflammatory response. In some embodiments, compositions utilized in the present invention become moldable at an elevated temperature into a pre-determined shape. Composites may become set when composites are implanted and allowed to cool to body temperature (approximately 37° C.).

The invention also provides methods of preparing inventive composites by combining tissue components and components and polyurethane precursors and resulting in naturally flowable compositions. Alternatively or additionally, the invention provides methods to make a porous composite include adding a solvent or pharmaceutically acceptable excipient to render a flowable or moldable composition. Such a composition may then be injected or placed into the site of implantation. As solvent or excipient diffuses out of the composite, it may become set in place. In further embodiments, the composite can be deposited on a film or other material that can enhance cellular infiltration into the scaffold. For instance, some embodiments of composites comprise at least one side that is coated with a film (e.g., CMC film, starch film, or the like) and the film can configured to face the direction of a wound or the like. In some embodiments, having a film on at least one side of a composite can enhance cellular infiltration, at least initially, on that side of the composite.

Polymer processing techniques may also be used to combine components with a polyurethane or precursors (e.g., polyisocyanates and polyols). In some embodiments, a composition of polyurethane may be rendered formable (e.g., by heating or with a solvent) and combined with components by injection molding or extrusion forming. Alternatively, polyurethanes and tissue components and components may be mixed in a solvent and cast with or without pressure. For example, a solvent may be dichloromethane. In some embodiments, a composition of particle and polymer utilized in the present invention is naturally injectable or moldable in a solvent-free condition.

In some embodiments, components may be mixed with a polymer precursor according to standard composite processing techniques. For example, regularly shaped components may simply be suspended in a precursor. A polymer precursor may be mechanically stirred to distribute the components or bubbled with a gas, preferably one that is oxygen-, and moisture-free. Once components of a composition are mixed, it may be desirable to store it in a container that imparts a static pressure to prevent separation of the components and the polymer precursor, which may have different densities. In some embodiments, distribution and particle/polymer ratio may be optimized to produce at least one continuous path through a composite along components.

Interaction of polymer components with tissue components and components may also be enhanced by coating individual components with a polymer precursor before combining them with bulk precursors. The coating enhances the association of the polymer component of the composite with the components. For example, individual components may be spray coated with a monomer or prepolymer. Alternatively, the individual components may be coated using a tumbler—components and a solid polymer material are tumbled together to coat the components. A fluidized bed coater may also be used to coat the components. In addition, the components may simply be dipped into liquid or powdered polymer precursor. All of these techniques will be familiar to those skilled in the art.

In some embodiments, it may be desirable to infiltrate a polymer or polymer precursor into vascular and/or interstitial structure of tissue components or into tissue-derived tissues. Vascular structure of tissue includes such structures for example the hepatic or renal vessels. Many of monomers and precursors (e.g., polyisocyanate prepolymers, polyols) suggested for use with the invention are sufficiently flowable to penetrate through the channels and pores. Thus, it may be necessary to incubate tissue components and components in polyurethane precursors for a period of time to accomplish infiltration. In certain embodiments, polyurethane itself is sufficiently flowable that it can penetrate channels and pores of tissue. Other ceramic materials and/or other tissue-substitute materials employed as a particulate phase may also themselves include pores that can be infiltrated as described herein.

Inventive composites utilized in the present invention may include various ratios of polyurethane and any other component, for example, between about 0 wt % and about 95 wt % other components. In some embodiments, composites may include about 10 wt % to about 15 wt % other components, about 15 wt % to about 20 wt % other components, about 20 wt % to about 25 wt % other components or about 25 wt % to about 30 wt % other components. In some embodiments, composites may include about 30 wt % to about 35 wt % other components. In some embodiments, composites may include at least approximately 10 wt %, 15 wt %, 20 wt %, 25 wt %, 30 wt %, or 35 wt %, 40 wt %, or 45 wt %, 50 wt %, or 55 wt %, 60 wt %, or 65 wt %, 70 wt %, or 75 wt %, 80 wt %, or 85 wt % of other components. In certain embodiments, such weight percentages refer to weight of other components, and may include biologicals, polysaccharides (e.g., tissue component), or any of the other components discussed above.

More specifically, some embodiments comprise about 5 wt % of a tissue component, which optionally may be a polysaccharide. Some embodiments comprise about 10 wt %, about 20 wt %, about 30 wt %, about 40 wt %, about 50 wt %, about 60 wt %, about 70 wt %, about 80 wt %, about 90 wt %, about 95 wt %, or any percentage therebetween of such tissue component.

Desired proportion may depend on factors such as injection sites, shape and size of the components, how evenly polymer is distributed among components, desired flowability of composites, desired handling of composites, desired moldability of composites, and mechanical and degradation properties of composites. The proportions of polymers and components can influence various characteristics of the composite, for example, its mechanical properties, including fatigue strength, the degradation rate, and the rate of biological incorporation. In addition, the cellular response to the composite will vary with the proportion of polymer and components. In some embodiments, the desired proportion of components may be determined not only by the desired biological properties of the injected material but by the desired mechanical properties of the injected material. That is, an increased proportion of components will increase the viscosity of the composite, making it more difficult to inject or mold. A larger proportion of components having a wide size distribution may give similar properties to a mixture having a smaller proportion of more evenly sized components.

Inventive composites of the present invention can exhibit high degrees of porosity over a wide range of effective pore sizes. Thus, composites may have, at once, macroporosity, mesoporosity and microporosity. Where only a porogen is present in the PUR scaffold, however, the initial porosity may be 0%. Macroporosity is characterized by pore diameters greater than about 100 microns. Mesoporosity is characterized by pore diameters between about 100 microns about 10 microns; and microporosity occurs when pores have diameters below about 10 microns. In some embodiments, the composite has a an initial porosity of at least about 30%. For example, in certain embodiments, the composite has a porosity of more than about 50%, more than about 60%, more than about 70%, more than about 80%, or more than about 90%. In some embodiments, inventive composites have a porosity in a range of 70%-80%, 80%-85%, or 85%-90%. Advantages of a porous scaffold over non-porous scaffold include, but are not limited to, more extensive cellular and tissue in-growth into the composite, more continuous supply of nutrients, more thorough infiltration of therapeutics, and enhanced revascularization, allowing tissue growth and repair to take place more efficiently. Furthermore, in certain embodiments, the porosity of the composite may be used to load the composite with biologically active agents such as drugs, small molecules, cells, peptides, polynucleotides, growth factors, etc, for delivery at the implant site. Porosity may also render certain composites of the present invention compressible.

The porosity of the cured scaffolds may vary from 30-70%, and the pore size may range from 177-700 μm or from 320-370 μm. When specific embodiments of PUR scaffolds are injected into 3-mm femoral condylar plug defects in rats, the composites may exhibit cellular infiltration and new bone formation at 3 weeks. Studies have shown that embodiments of pre-formed PUR scaffolds implanted in both subcutaneous¹⁴ and excisional¹⁵ wounds in Sprague-Dawley rats supported cellular infiltration and ingrowth of new tissue.

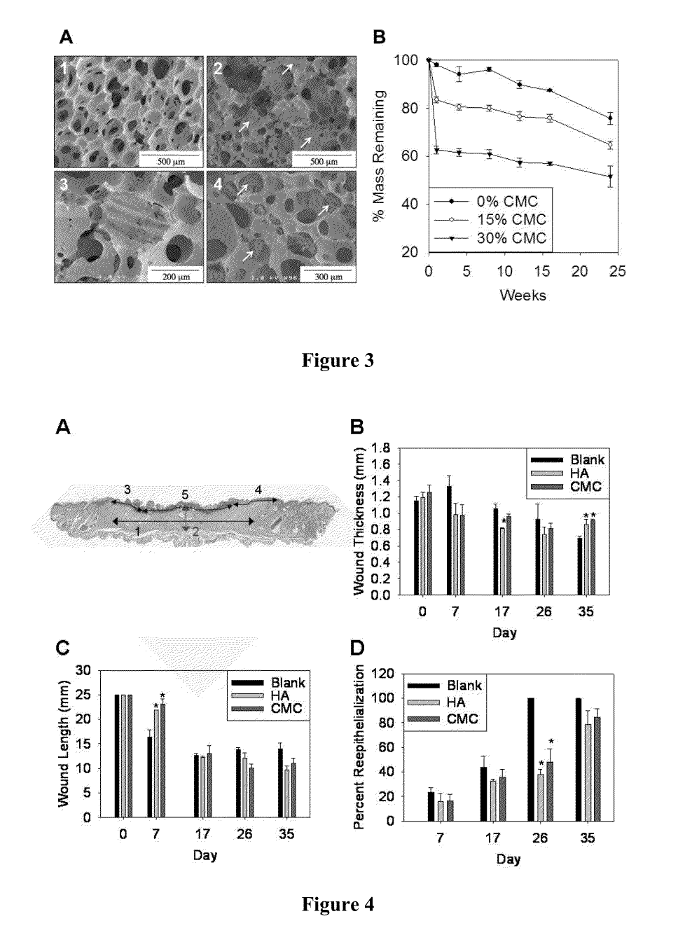

Certain embodiments of PUR scaffolds may exhibit pore sizes ranging from 320-370 μm, which may be comparable to those that may facilitate infiltration of cells such as fibroblasts (90-360 μm²⁶) and osteoblasts.³² Embodiments comprising polysaccharides, for example HA and/or CMC, may exhibit higher density and modulus and lower porosity than non-polysaccharide PUR scaffolds. However, embodiments of PUR scaffolds may be designed so that after seven days of applying a treatment the density, modulus, and porosity of polysaccharide-filled scaffolds are not significantly different than those for PUR scaffolds without polysaccharide filler. See, for example, the SEM images of specific embodiments in FIG. 3A and the degradation data in FIG. 3B that suggest, without being bound by theory or mechanism, polysaccharides leach from the scaffolds by day 7 following surgery, which may result in lower modulus and density. Embodiments of PUR scaffolds incorporating 7-8% tobramycin also may exhibit similar characteristics 7 days following surgery.³³

In some embodiments, pores of inventive composite may be over 100 microns wide for the invasion of cells and tissue in-growth (Klaitwatter et al., J. Biomed. Mater. Res. Symp. 2:161, 1971; incorporated herein by reference). In certain embodiments, the pore size may be in a range of approximately 50 microns to approximately 1000 microns, for example, of approximately 100 microns to approximately 500 microns. In some embodiments, compressive strength of dry scaffolds may be in an approximate range of 17−97 kPa, while compressive modulus may be in an approximate range of 25−216 kPa. After implantation, inventive composites are allowed to remain at the site providing the strength and modulus desired while at the same time promoting healing of the tissue and/or tissue growth. Polyurethane of composites may be degraded or be resorbed as new tissue is formed at the implantation site. Polymer may be resorbed over approximately 2 weeks to approximately 2 years. Composites may start to be remodeled in as little as a week as the composite is infiltrated with cells or new tissue in-growth. A remodeling process may continue for weeks, months, or years. For example, polyurethanes used in accordance with the present invention may be resorbed within about 4-8 weeks, 2-6 months, 6-12 months, 12-18 months, or 18-24 months. A degradation rate is defined as the mass loss as a function of time, and it can be measured by immersing the sample in phosphate buffered saline or medium and measuring the sample mass as a function of time.

One skilled in the art will recognize that standard experimental techniques may be used to test these properties for a range of compositions to optimize a composite for a desired application. For example, standard mechanical testing instruments may be used to test the compressive strength and stiffness of composites. Cells may be cultured on or transplanted as part of composites for an appropriate period of time, and metabolic products and amount of proliferation (e.g., the number of cells in comparison to the number of cells seeded) may be analyzed. Weight change of composites may be measured after incubation in saline or other fluids. Repeated analysis will demonstrate whether degradation of a composite is linear or not, and mechanical testing of incubated materials will show changes in mechanical properties as a composite degrades. Such testing may also be used to compare enzymatic and non-enzymatic degradation of a composite and to determine levels of enzymatic degradation. A composite that is degraded is transformed into living tissue upon implantation or transplantation from cell/tissue culture or bio-reactor.

Use and Application of Composite

As discussed above, polymers or polymer precursors, tissue components, and other components may be supplied separately, e.g., in a kit, and mixed immediately prior to implantation, injection or molding. A kit may contain a preset supply of tissue and/or other components having, e.g., certain sizes, shapes, and physical form. Surface of tissue components and other components may have been optionally modified using one or more of techniques described herein. Alternatively, a kit may provide several different types of components of varying sizes, shapes, and levels of demineralization and that may have been chemically modified in different ways. A surgeon or other health care professional may also combine components in a kit with autologous tissue and components derived during surgery or biopsy. For example, a surgeon may want to include autogenous tissue or cells, (e.g., marrow or tissue grafts) generated while preparing an implant site, into a composite.

Composites of the present invention may be used in a wide variety of clinical applications. A method of preparing and using polyurethanes for orthopedic applications utilized in the present invention may include the steps of providing a curable tissue/PUR composition, mixing parts of a composition, and curing a composition in a tissue site wherein a composition is sufficiently flowable to permit injection by minimally invasive techniques. In some embodiments, a flowable composition to inject may be pressed by hand or machine. In some embodiments, a moldable composition may be pre-molded and implanted into a target site. Injectable or moldable compositions utilized in the present invention may be processed (e.g., mixed, pressed, molded, etc.) by hand or machine. These mixing techniques provides significant advantages over the previous method of mixing in a large bench top mixer, such that they are self-contained, portable, and can be easily used and customized in the surgical operating room without additional equipment.

Certain embodiments of composites and/or compositions may be used as injectable materials with or without exhibiting high mechanical strength (i.e., load-bearing or non-load bearing, respectively). In some embodiments, inventive composites and/or compositions may be used as moldable materials. For example, compositions (e.g., prepolymer, monomers, reactive liquids/pastes, polymers, tissue components and other components, etc.) in the present invention can be pre-molded into pre-determined shapes. Upon implantation, the pre-molded composite may further cure in situ and may or may not provide tissue specific functional mechanical strength (i.e., load-bearing). For instance, the composite may be molded into the shape of a graft, and then the graft can be deposited on a wound or generally any surface outside or inside the body of a subject.

Exemplary PUR composites can be useful for a variety of applications, including, but not limited to, injectable scaffolds for wound healing and drug and gene delivery. Some composites can, for example, be applied to a wound site or surface. The composites can be injected through the skin of a patient to, for example, fill a void, cavity, or hole created by a wound using, for example, a syringe. In some embodiments, compositions and/or composites of the present invention may be used as a tissue void filler. Tissue defects, which result from trauma, injury, infection, malignancy or developmental malformation can be difficult to heal in certain circumstances. If a defect or gap is larger than a certain critical size, natural tissue is unable to bridge or fill the defect or gap. These are several deficiencies that may be associated with the presence of a void in a tissue. A tissue void may compromise mechanical integrity of the tissue, making the tissue potentially susceptible to dehiscence or chronic infection or inflammation until the void becomes ingrown with native tissue. Accordingly, it is of interest to fill such voids with a substance which helps voids to eventually fill with naturally or endogenously generated tissue. Open defects in practically any tissue may be filled with composites according to various embodiments. Even where a composite is not required to support full function; physiological forces will tend to encourage remodeling of a composite to a shape reminiscent of original tissues.

In certain embodiments, it is the physical, mechanical, and rheological properties of the PUR composites that may render them suitable for use as injectable scaffolds in the setting of cutaneous wound repair. Certain embodiments of PUR scaffolds may be designed to have working and cure times of, respectively, less than 7 and 19 minutes, and more specifically, within 5-7 and 15-19 minutes. These setting and working times, which may be altered depending on the needs of a particular application, may be compatible with the temporal limitations imposed by the clinical setting. Certain embodiments may exhibit compressive properties that approach those of intact skin, and thus the scaffolds may stent wounds at early time points and promote granulation tissue formation while preventing wound contraction. Without being bound by theory or mechanism, embodiments of PUR scaffolds may allow for collagen synthesis and organization, as well as myofibroblast formation, which may yield a net positive impact on wound healing.

In this regard, embodied composites can be used in a large variety of clinical applications. For example, some embodiments can be used as soft tissue (i.e., non osseous tissue) void fillers, to repair or help healing of tissue or organ deficiencies resulting from trauma, tumors, surgery, iatrogenic, congenital, genetic, metabolic and degenerative or abnormal development, and inflammatory infection. In some embodiments, inventive composites promote cellular infiltration from adjacent tissues, thus accelerating the remodeling process. The composites may be used for the repair of a simple, complex, tissue void or tissue augmentation or tissue obliteration, for reconstruction, or repair or therapeutic delivery to the integument, subdermal tissue, breast tissue, vascular tissue, cardiac tissue, urogential-renal tissue, pulmonary tissue, hepatic tissue, gastrointestinal tissue, muscle tissue, ligament tissue, tendon tissue, facial tissue, gynecologic and female reproductive genital tissue, non-articular surface fibrocartilage tissue and cartilage tissue and special sensory tissues and neural tissue. Those of ordinary skill will appreciate that the term “treating a wound” and the like, as used herein, refers at least to the treatment (e.g, healing) of any of the above-described deficiencies that may be on any of the tissues described here.

Proliferative assays of certain embodiments of PUR scaffolds demonstrate that the scaffolds may support cellular attachment and proliferation, indicating that a scaffold may be non-toxic and biocompatible as it degrades and is replaced by new matrix. In specific embodiments of PUR, PUR+HA, and PUR+CMC scaffolds, no significant differences in the level of apoptosis was noted, which without being bound by theory or mechanism, may suggest that the PUR scaffolds and their degradation products are noncytotoxic and do not harm the surrounding tissue.

Many soft tissue defects are created in surgery for trauma, oncology, and aesthetic procedures. One example is in breast surgery for cancer whereby a “lumpectomy” is performed. The size of the defect can be quite significant and impact body symmetry—the use of the invention to fill the defect and have the tissue component encourage regeneration of adipose type tissue of equivalent differentiated and mechanical functional tissue is desired. In oncology there may be metastatic cancer deposits in bone or liver, and these lesions can be therapeutically addressed by treatment with the composite of the invention containing a therapeutic agent that is slowly released locally. During aging there is thought to be a loss of subdermal tissue volume and the invention can be used for augmentation restoration of the facial area. (see Coleman-Fat transplantation).

Many orthopedic, periodontal, neurosurgical, oral and maxillofacial surgical procedures require drilling or cutting into tissue in order to harvest autologous implants used in procedures or to create openings for the insertion of implants. In either case voids are created in tissues. In addition to all the deficiencies associated with tissue void mentioned above, surgically created tissue voids may provide an opportunity for incubation and proliferation of any infective agents that are introduced during a surgical procedure. Another common side effect of any surgery is ecchymosis in surrounding tissues which results from bleeding of the traumatized tissues. Finally, surgical trauma to tissue and surrounding tissues is known to be a significant source of post-operative pain and inflammation. Surgical tissue voids are sometimes filled by the surgeon with autologous tissue chips that are generated during trimming of bony ends of a graft to accommodate graft placement, thus accelerating healing. However, the volume of these chips is typically not sufficient to completely fill the void. Composites and/or compositions of the present invention, for example composites comprising anti-infective and/or anti-inflammatory agents, may be used to fill surgically created tissue voids.

Inventive composites may be administered to a subject in need thereof using any technique known in the art. A subject is typically a patient with a disorder or disease related to tissue. In certain embodiments, a subject has a tissue defect such as an open skin wound or cut. Any tissue disease or disorder may be treated using inventive composites/compositions including genetic diseases, open sores, wounds, cuts, scrapes, and the like. In some embodiments the disease or disorder, such as a wound, is worsened by the presence of a second disease or disorder, such as diabetes.

Composites and/or compositions of the present invention can be used as tissue void fillers either alone or in combination with one or more other conventional devices, for example, to fill the space between a device and tissue. Examples of such devices include, but are not limited to, tissue fixation plates, screws, tacks, clips, staples, nails, pins or rods, anchors (e.g., for suture, tissue, and the like), scaffolds, scents, stitches, bandages, meshes (e.g., rigid, expandable, woven, knitted, weaved, etc), sponges, implants for cell encapsulation or tissue engineering, drug delivery (e.g., carriers, tissue ingrowth induction catalysts such as tissue morphogenic proteins, growth factors (e.g., PDGF, VEGF and BMP-2), peptides, antivirals, antibiotics, etc), monofilament or multifilament structures, sheets, coatings, membranes (e.g., porous, microporous, resorbable, etc), foams (e.g., open cell or close cell), screw augmentation, cranial, reconstruction, and/or combinations thereof.

Certain embodiments of degradable PUR scaffolds may function as an initial temporary matrix that, without being bound by theory or mechanism, provides a surface for attachment and proliferation of cells and also stents the wound, potentially minimizing the undesirable outcomes of contraction and scarring, which may be caused by cells within and surrounding a scaffold or implant. Embodiments of the injectable PUR networks may be rubbery elastomers at physiological temperatures with glass transition temperatures (T_{g}) less than 10° C., and they may sustain compressive strains exceeding 50% without mechanical failure.¹⁴ Data collected from certain embodiments of wound healing, cell proliferation, and matrix deposition indicate that PUR scaffolds may delay contraction and scarring. See, for example, FIGS. 4-9.

In certain embodiments it may be advantageous, and contraction, scarring, and the like may be minimized, by implementing a PUR scaffold with a Young's modulus that when measured under compressive deformation is comparable to that of skin from a patient, including humans and other animals. For example, the Young's modulus of certain embodiments of PUR scaffolds measured under compressive deformation approaches that of human skin, which has been reported as 35 kPa for the dermis³⁷, and rat skin, which has been measured to be 400±150 kPa.

Without being bound by theory or mechanism, cutaneous wound repair goes through predictable stages, characterized by an initial acute inflammatory phase that leads to ingrowth of granulation tissue followed by a progressive transition to sustained matrix production and remodeling. Rapid wound closure often leads to excessive matrix production and the very undesirable outcomes of scarring and wound contraction, which were not observed with treatments done with embodiments of PUR scaffolds. Specific embodiments may allow for matrix production to be visibly dampened and the alignment of collagen fibers to be more random compared to wounds not treated with PUR scaffolds. Thus, embodiments of PUR scaffolds may resist the contractile forces that are generated in the host tissue, and may promote cellular infiltration and remodeling rather than excessive matrix deposition and scarring.

Looking to FIG. 4, excisional wounds treated with an embodiment of PUR scaffolds indicate that embodiments of PUR scaffolds may stent the wounds at early time points, thus leading to a restorative rather than a scarring/contracting phenotype at later time points. Furthermore, myofibroblasts may generate unwanted contractile forces that promote wound contraction and fibrosis. The architectural disruption of myofibroblast alignment caused by treatment with embodiments of PUR scaffolds may lead to a more reticular arrangement of collagen fibers. Even in embodiments wherein the upper surface of the PUR scaffolds was approximately flush with the surface of the skin, epidermal resurfacing of the wounds may delayed. Thus, delays in re-epithelization and the effects on myofibroblast accumulation and orientation may be potentially advantageous features of embodiments of the present invention.

Longitudinal studies of certain embodiments of PUR scaffolds showed a marked difference in the alignment of collagen fibers and cells within the PUR scaffolds. Without being bound by theory or mechanism, it is thought that that the transient presence of scaffolds may disrupt the formation of uniformly aligned extracellular matrix under elevated tension. In certain embodiments, the PUR scaffold degrades at a rate comparable to that of new tissue ingrowth.

Certain embodiments of lysine-derived PUR scaffolds may undergo oxidative degradation to soluble break-down products mediated by macrophages in vivo.¹⁷ Scaffolds may be almost completely resorbed after 4 weeks post-implantation in rat excisional wounds.¹⁷ Biostable PUR foams have been developed as coverings to minimize fibrous encapsulation of breast implants.^{38,39} However, PUR foams may slowly degraded in vivo into small pieces after periods longer than 18 months post-implantation, thereby inducing fibrous encapsulation of the implant and an intense foreign-body response to the foam fragments. The delayed appearance of myofibroblasts in the injectable scaffolds may be consistent with an altered mechanical environment, particularly in light of the evidence that cell-generated tension in the context of relatively stiff extracellular matrix may lead to the activation of latent TGF-β, which promotes matrix accumulation and differentiation of the myofibroblast phenotype.⁴⁰

Embodiments of injectable PUR scaffolds may accelerate wound healing through the local delivery of biologics such as recombinant human platelet-derived growth factor (rhPDGF)¹⁵, antibiotics^{33,41}, and the like. Delivery of rhPDGF-BB from embodiments of PUR scaffolds implanted in excisional wounds in rats may accelerate both ingrowth of new tissue and/or degradation of the scaffolds.¹⁵ Delivery of vancomycin from embodiments of PUR scaffolds implanted in a contaminated femoral segmental defect in rats may decrease bacterial counts in both bone and soft tissue.⁴¹ Biologics may be added to the polyester triol component prior to mixing with the prepolymer, thereby facilitating clinical ease of use and customization at the point of care.

### EXAMPLES

The following non-limiting example represents descriptions of certain embodiments of the present invention and experimentation methods that are meant to serve illustrative purposes and that shall not limit the present invention in any manner. For the Examples below, where applicable, single factor analysis of variance (ANOVA) was used to evaluate the statistical significance of results. For the data collected, P values over 0.05 may be labeled with an asterisk on the corresponding charts to indicate statistically significant values.

### Example 1

This Example describes the preparation and synthesis of PUR foams in accordance with embodiments of the present invention.

Various materials were used in the preparation and synthesis of the PUR foams. Glycolide and D,L-lactide were purchased from Polysciences (Warrington, Pa.). TEGOAMIN33, a tertiary amine catalyst composed of 33 wt % triethylene diamine (TEDA) in dipropylene glycol, was obtained from Goldschmidt (Hopewell, Va.). Polyethylene glycol (PEG, 200 Da) was supplied by Alfa Aesar (Ward Hill, Mass.). Glycerol and the sodium salts of carboxymethyl cellulose (CMC; 90-kDa) and hyaluronic acid (HA; 1,500-2,200-kDa) were purchased from Acros Organics (Morris Plains, N.J.). Lysine triisocyanate (LTI) was obtained from Kyowa Hakko USA (New York), and stannous octoate catalyst was obtained from Nusil technology (Overland Park, Kans.). All other reagents were purchased from Sigma-Aldrich (St. Louis, Mo.). Prior to use, glycerol and PEG were dried at 10 mm Hg for 3 h at 80° C., and ε-caprolactone was dried over anhydrous magnesium sulfate. All other materials were used as received.

To synthesize the PUR foams, reactive intermediates were first synthesized. PEG (200 Da) was reacted with an excess of LTI (NCO:OH equivalent ratio=3:1) to form an LTI-PEG prepolymer in which the PEG molecules were end-capped with LTI.¹³ PEG was added dropwise to LTI in a 100 mL reaction flask with stirring under argon for 24 h at 45° C. The prepolymer was then dried under vacuum at 80° C. for 14 h. A polyester triol (900 Da) with a backbone comprising 60% caprolactone, 30% glycolide, and 10% lactide was synthesized by reacting the monomers (ε-caprolactone, glycolide, and D,L-lactide) with a glycerol starter in the presence of stannous octoate catalyst.¹⁶ This polyester triol composition and molecular weight may maintain both good flowability of the reactive mixture as well as a favorable degradation rate of the cured PUR scaffold in vivo.¹⁷ The reaction was carried out under dry argon at 140° C. for 48 h, and the resulting polyester triol was dried under vacuum at 80° C. for 24 h.

PUR scaffolds were then synthesized by reactive liquid molding of the LTI-PEG prepolymer with a hardener component^{13,14} and a polysaccharide filler (carboxymethyl cellulose [CMC] or hyaluronic acid [HA]). The hardener comprised 100 parts polyester triol (polyol), 1.5 parts per hundred parts polyol (pphp) water, 0.625 pphp TEGOAMIN33 catalyst, 0.375 pphp 30% bis(2-dimethylaminoethyl)ether (DMAEE) blowing catalyst in poly(propylene glycol), and 4.0 pphp calcium stearate pore opener. The polysaccharide was combined with the hardener and mixed by hand for 30 s. The prepolymer was added to the hardener and polysaccharide and mixed by hand for 1 min. The resulting mixture then rose freely for 10-20 min and cured. The targeted index (the ratio of NCO to OH equivalents times 100) was 115.

### Example 2

This Example describes the kinetics involved in the synthesis of the PUR scaffolds of Example 1 as well as possible considerations that may be used to optimize a PUR scaffold to meet the limitations of a particular circumstance.

The reactivities, or the specific reaction rates, for the second order reactions of the LTI-PEG prepolymer with the polyester triol, water, HA, and CMC were measured using attenuated total reflectance fourier transform infrared spectroscopy (ATR-FTIR; Bruker Tensor 27 FTIR, Billerica, Mass.). Prepolymer; TEGOAMIN33 and DMAEE catalysts; and either polyol, HA, or CMC were mixed together for 1 min and then placed in contact with the ATR crystal. The area of the isocyanate peak (wavelength 2150-2350 cm) was monitored as a function of time.

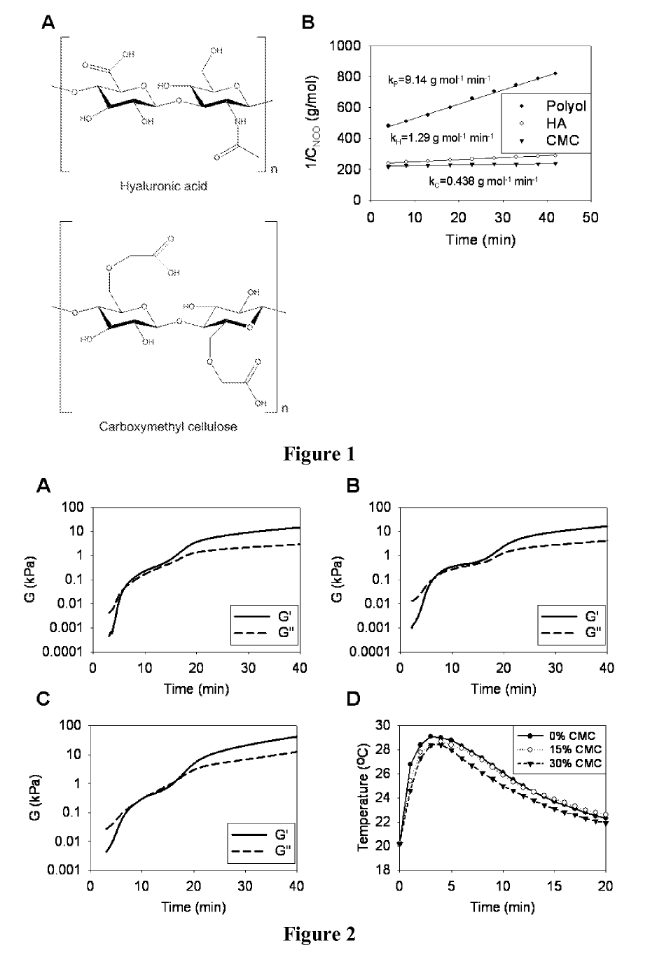

Looking to FIG. 1B, the results of the reactivity studies are shown. Although not shown in FIG. 1B, water may be the most reactive, and may have a rate constant of 600 g mol⁻¹ min⁻¹. For a certain embodiment, the rate constant measured for polyester triol (9.14 g mol⁻¹ min⁻¹) may be 21 times larger than that measured for CMC (0.438 g mol⁻¹ min⁻¹) and 7 times larger than that measured for HA (1.29 g mol⁻¹ min⁻¹). These data indicate that the water and polyester triol components may be the most reactive in the system and considerably more reactive than the polysaccharides. The higher reactivity of HA compared to CMC may be attributed to their structures, which are shown in FIG. 1A. Specifically, each repeat unit of HA has one primary OH group, whereas CMC has only carboxylic acids and secondary OH groups.

### Example 3

This Example describes the rheological properties of PUR scaffolds, such as those of Example 1, during cure. This Example provides insight of how to adjust working and tack-free times for the foams to meet the limitations of particular circumstances. The temperature data indicate that embodiments of foams may be suitable for in vivo applications.

The cure profiles of the HA and CMC scaffolds were measured using a TA Instruments parallel plate AR 2000ex rheometer operating in dynamic mode with 25 mm disposable aluminum plates (New Castle, Del.). LTI-PEG prepolymer was added to a mixture of hardener and polysaccharide (0, 15, or 30 wt %) and mixed by hand using a spatula for 1 min. The sample was then loaded onto the bottom plate of the rheometer. An oscillation time sweep was run with a controlled strain of 1% and a frequency of 6.28 rad/s in order to obtain the cure profile of each PUR scaffold. The storage modulus (G′) and loss modulus (G″) were determined as a function of time. The working time was determined to be the G-crossover point. To measure the setting time, the surface of the foam was contacted with a spatula at regular intervals of 30 sec. The tack-free time, which approximates the setting time, was determined to be the time at which the foam did not stick to the spatula.

The compiled data showing the rheological properties of embodiments of PUR, PUR+CMC, and PUR+HA scaffolds are shown in FIG. 2(A-C). The G-crossover point may be considered to be the gel point and thus the working time of the foam. Looking to certain embodiments, the working time was 5.8±0.7 min for the PUR foam, 6.2±0.5 min for the PUR+CMC foam, and 5.5±0.6 min for the PUR+HA foam. It may be possible to adjust working time by, among other things, altering the concentrations of the catalysts. Catalyst amount was kept constant for the purposes of this Example. For the embodiments that produced the data shown in FIG. 2, the tack-free time was 16±3 min for the PUR foam, 19±3 min for the PUR+CMC foam, and 15±4 min for the PUR+HA foam.

Furthermore, temperature profiles of the reactive mixtures during foaming were measured using a digital thermocouple at the centers of the rising foams, which were insulated to minimize the effects of heat loss from the exterior surface. Turning to FIG. 2D, the temperature profiles of embodiments of PUR, PUR+CMC, and PUR+HA foams are shown. Starting at room temperature, the maximum increase in temperature was 7.3±1.7° C. for the PUR foam, 7.1±1.4° C. for the PUR+CMC foam, and 6.7±1.1° C. for the PUR+HA foam.

### Example 4

This Examples describes experiments conducted that characterize the scaffolds of Example 1. These characterizations may indicate what applications the scaffolds of Example 1 are suitable for, and specifically what tissue may infiltrate the scaffold. The data also indicate that the dissolution of polysaccharides affects a scaffold's physical characteristics.

Core densities and porosities were determined from mass and volume measurements of triplicate cylindrical foam cores.^{14,18} The scaffold pore size distribution was assessed by scanning electron microscopy (Hitachi S-4200 SEM, Finchampstead, UK) after gold sputter coating with a Cressington Sputter Coater. Physical properties of the PUR scaffolds before and after incubating in an aqueous environment for 7 days are shown in Table 1, shown below. On day 0, the properties of PUR+HA and PUR+CMC scaffolds were not significantly different from each other, but both had significantly higher densities (45%), lower porosities (4%), and smaller pore sizes (13%) than the blank PUR scaffolds. However, by day 7 there were no significant differences in porosity, pore size, or density between the three groups, which, without being bound by theory or mechanism, may have been due to the dissolution of the polysaccharides.

TABLE 1

| | Day 0 | | | Day 7 | | |
|---|---|---|---|---|---|---|
| | Density | Porosity | Pore Size | Density | Porosity | Pore Size |
| PUR Sample | (kg/m³) | (vol %) | (μm) | (kg/m³) | (vol %) | (μm) |
| Blank PUR | 110 ± 2 | 90.9 ± 0.1 | 370 ± 90 | 105 ± 2 | 914 ± 0.1 | 320 ± 70 |
| PUR + HA | 158 ± 9 | 87 ± 0.7 | 330 ± 70 | 100 ± 7 | 91.8 ± 0.6 | 330 ± 80 |
| PUR + CMC | 161 ± 8 | 86.7 ± 0.6 | 320 ± 80 | 116 ± 18 | 90.4 ± 1.5 | 340 ± 90 |

Physical properties of specific embodiments of PUR scaffolds.

### Example 5

This Example describes degradations studies conducted on the scaffolds of Example 1. The degradation data described below provides insight as to how such scaffolds may have the superior and unexpected benefit of biodegrading within a subject during treatment

Scaffold degradation was evaluated by incubating triplicate 20 mg samples in 1 ml phosphate buffered saline (PBS) (pH 7.4) at 37° C. for up to 24 weeks. At various time points, the samples were rinsed in deionized water, dried under vacuum for 48 h at room temperature, and weighed.

A SEM image of an embodiment of a PUR scaffold is shown in FIG. 3A.1. The interconnected pores of the scaffolds permit cellular infiltration.¹⁴ FIGS. 3A.2 and 3A.3 show images of 100-200 μm HA particles embedded in a PUR+HA scaffold at low and high magnification, respectively. As shown in FIG. 3A.4, the particles were almost completely dissolved after 24 h in vitro incubation time in buffer. Without being bound by theory or mechanism, CMC, HA, or other filler particles leach in moist environments, as would occur in vivo, which may create additional pores. Alcian blue staining was used to confirm the presence of HA particles embedded in the scaffolds. PUR (negative control) and PUR+HA scaffolds were stained with Alcian blue at pH 2.5 and pH 1.0. At pH 1.0, Alcian blue only stains highly sulfated glycosaminoglycans, while at pH 2.5 the dye stains HA blue. PUR scaffolds did not stain at either pH and PUR+HA scaffolds did not stain at pH 1.0, but PUR+HA scaffolds stained blue at pH 2.5, thereby confirming the presence of HA in the scaffolds.

To investigate the effects of polysaccharide loading on scaffold degradation, the degradation rates of PUR, PUR+15% CMC, and PUR+30% CMC in PBS at 37° C. were recorded for up to 24 weeks (FIG. 3B). Under in vitro conditions, and without being bound by theory or mechanism, the primary mechanism of degradation was hydrolysis of the ester bonds within the polyester soft segment.¹⁷ The polysaccharides may cause a high initial mass loss within the first few days, which is consistent with the SEM data shown in FIG. 3A.4. After this time period, the rates of polymer degradation for these specific embodiments were similar.

### Example 6

This Example describes the thermal and mechanical properties of the scaffolds of Example 1.

Thermal transitions of the materials were evaluated by differential scanning calorimetry (DSC) using a Thermal Analysis Q10000 DSC. 10 mg samples underwent two cycles of cooling (20° C./min) and heating (10° C./min), between −80° C. and 100° C. Mechanical properties were measured using a TA Instruments Q800 Dynamic Mechanical Analyzer (DMA) in compression mode (New Castle, Del.). Samples were tested either shortly after fabrication or after 7 days of incubation in PBS prior to mechanical testing. Stress-strain curves were generated by compressing wet cylindrical 7×6 mm samples at 37° C. at a rate of 0.1 N/min until they reached 50% strain. The Young's modulus was determined from the slope of the initial linear region of each stress-strain curve. The scaffolds could not be compressed to failure due to their elasticity, so the compressive stress was measured one minute after the application of 50% strain.¹⁴

The compressive Young's modulus and compressive stress of embodiments of PUR scaffolds under physiological conditions (e.g. wet at 37° C.) before and after incubation for 7 days are summarized in Table 2, shown below.

TABLE 2
Mechanical properties of embodiments of PUR scaffolds

| | Day 0 | | | |
|---|---|---|---|---|
| | Young's | | Day 7 | |
| | Modulus | Compressive | Young's | Compressive |
| PUR Sample | (kPa) | Stress (kPa) | Modulus (kPa) | Stress (kPa) |
| Blank PUR | 30 ± 4 | 7.7 ± 1.0 | 19 ± 8 | 6.8 ± 0.6 |
| PUR + HA | 50 ± 20 | 10 ± 2 | 11 ± 4 | 8 ± 3 |
| PUR + CMC | 60 ± 30 | 10 ± 7 | 14 ± 4 | 9 ± 3 |

When compressed for extended periods of time, the embodied PUR scaffolds exhibited less than 5% permanent deformation, which is consistent with the properties of rubbery elastomers. Furthermore, the materials did not fail under compression, so compressive stress-strain tests were carried out to 50% strain, where the compressive stress was measured as reported previously.²⁰ The initial modulus and strength of scaffolds containing polysaccharide filler were higher, but not significantly, than those of blank PUR scaffolds. After incubating in PBS for 7 days, the modulus and strength of all three scaffolds decreased, but only the changes in the modulus of the polysaccharide-filled scaffolds were significant (p<0.005 for PUR+HA and p<0.02 for PUR+CMC).

### Example 7

Example 7 describes in vivo cutaneous repair in rats using the scaffolds of Example 1. Using an excisional wound model, this Example analyzes the effects of PUR scaffolds on the measurement of wounds, proliferation and apoptosis of cells, wound contraction, and collagen production.

All surgical procedures for this Example were reviewed and approved by the local Institutional Animal Care and Use Committee. NIH guidelines for the care and use of laboratory animals (NIH Publication #85-23 Rev. 1985) have been observed. The capacity of the scaffolds to facilitate dermal wound healing was evaluated in an excisional wound model (6.25 cm² square wounds) in adult male Sprague-Dawley rats. All materials were sterilized by gamma irradiation at 5 kGy prior to surgery. The treatment groups investigated were untreated wounds (negative control), PUR+15 wt % HA scaffolds, and PUR+15 wt % CMC scaffolds. For the HA and CMC treatment groups, the materials were applied as a reactive liquid immediately after mixing the LTI-PEG prepolymer with the hardener and polysaccharide (15 wt % CMC or HA). The PUR expanded by gas foaming to fill the defects and cured in situ. When the scaffolds expanded beyond the wound dimensions, they were trimmed to be flush with the skin surface. Each wound and scaffold was covered with nonadherent, absorbent, Release gauze (Johnson & Johnson) and covered with a Tegaderm outer dressing (3M, St. Paul, Minn.). Wounds were harvested at days 7, 17, 26, and 35 after surgery. Four replicates of each treatment group were harvested at each time point. The wounds were fixed in neutral buffered formalin for 24 h, transferred into 70% ethanol for 48 h, embedded in paraffin, and sectioned at 5 μm. Hematoxylin & eosin (H&E), Gomori's trichrome, picrosirius red, TUNEL, myeloperoxidase, Ki67, α-SMA, and procollagen I immunostaining were performed on the tissue sections.

A. Measurement of Excisional Wounds

Embodiments of injectable PUR scaffolds with 15% CMC or HA were tested for their effects upon dermal wound healing in a rat excisional wound model. No frank necrosis of the surrounding tissue was seen at the early time points, suggesting that the mild exotherm resulting from the PUR reaction may not adversely affect the host tissue. Also, the level of apoptosis in the scaffold-treated groups may be similar to that of blank wounds (FIG. 4B). The average length in the longitudinal direction (i.e. the direction of contraction), granulation tissue thickness, and percent re-epithelialization of the wounds in the three treatment groups at each time point are summarized in FIG. 4(B-D). FIG. 4(A) shows a schematic for how these values were ascertained. At days 7 and 17, the thickness of the wounds in the HA and CMC treatment groups was less than the thickness of the blank wounds; however, only the thickness of the wounds in the HA group at day 17 was significantly less than the blank (p<0.015). At day 7, the length of the blank wounds was less than those of the HA and CMC groups (p<0.045, p<0.015, respectively), providing evidence that the PUR scaffolds stented the wound. Blank contracted wounds were fully epithelialized by day 26, while HA and CMC treatment groups stented were not fully epithelialized by day 35.

B. Cell Profileration and Apoptosis

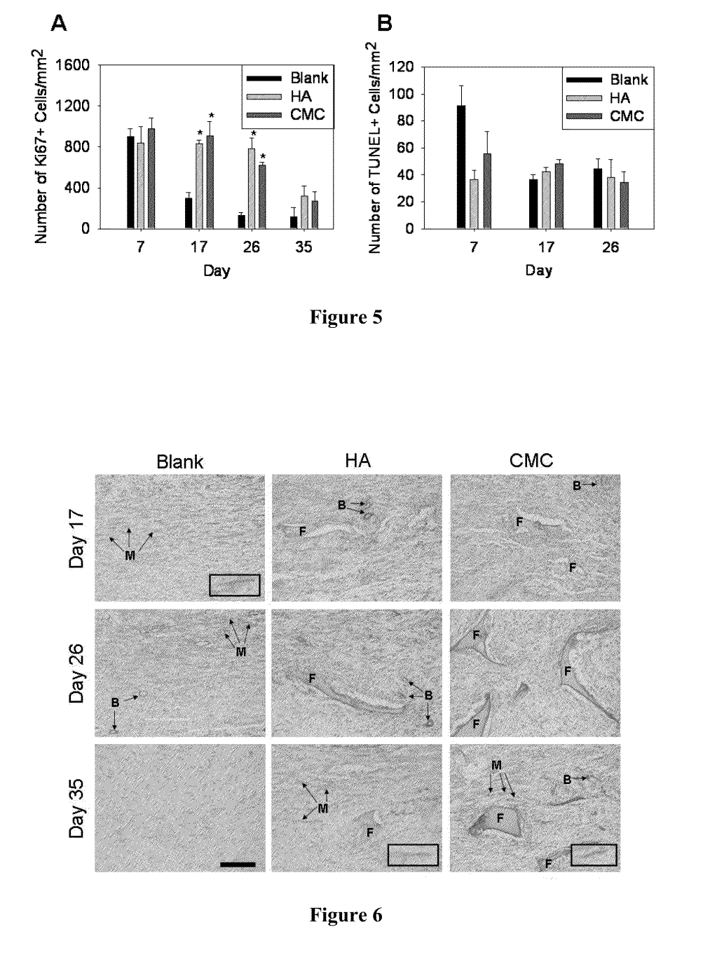

Ki67 staining was performed to assess the level of cell proliferation within the wound bed (FIG. 5A). After 7 days, we found no difference in the number of Ki67⁺ cells in the blank wounds compared to the scaffold treatment groups. From day 7 to day 17, the number of proliferating cells remained constant in the CMC and HA treatment groups but decreased by 67% in the blank treatment group. Thus at day 17, the number of Ki67⁺ cells was significantly higher in the scaffold treatment groups than in the blanks. The number of Ki67⁺ cells decreased slightly from day 17 to day 26, but the level of proliferation in the scaffold treatment groups remained significantly higher than in the blank wounds. From day 26 to day 35, the number of Ki67⁺ cells decreased by 40% in the scaffold treatment groups and remained constant in the blank treatment group. At day 35, the number of Ki67⁺ cells in the scaffold treatment groups was comparable to that observed for the blank wounds.

TUNEL staining was used to measure cell apoptosis in the wound site (FIG. 5B). At day 7, the number of cells stained with TUNEL was higher in the blank wounds than in the wounds with PUR scaffolds, but the difference was not statistically significant. From day 7 to day 17, the number of cells stained with TUNEL decreased by 40% in the blank wounds and remained relatively constant in the scaffold treatment groups. The level of apoptosis did not change in any of the treatment groups from day 17 to day 26. There were no significant differences in the number of cells stained with TUNEL among the three treatment groups at any of the time points.

C. Contraction

Staining for α-smooth muscle actin (α-SMA) was performed in order to examine the formation of myofibroblasts in the wound site. Representative images of sections stained for α-SMA are displayed in FIG. 6. In the blank wounds, the number of myofibroblasts was greatest at days 17 and 26 and decreased almost completely by day 35. In contrast, fewer myofibroblasts were present at days 17 and 26 in the HA and CMC treatment groups. Myofibroblast formation in these groups was delayed and remained higher at the day 35 interval than in the blank group. Myofibroblasts were oriented parallel to the epidermis in the blank wounds, forming lines of tension in the skin as is characteristic of wounds undergoing scarring and contraction. In contrast, myofibroblasts were randomly oriented around pieces of PUR in the PUR+HA and PUR+CMC treatment groups. These results show that myofibroblast formation may be delayed in the PUR+HA and PUR+CMC groups due to fragments of PUR scaffolds that may disrupt the linear alignment of myofibroblasts.

Without being bound by theory or mechanism, during the nascent phases of cutaneous wound repair, the provisional loose connective tissue matrix develops a very robust capillary network, which causes the healing wound to appear red due to the fragile capillaries that bleed easily. If healing progresses through its expected phases, the number of new capillaries peaks and subsequently begins to decline. By days 26 and 35 in the life of the wound, the capillary density is regressing, which is consistent with the histological sections in FIG. 6. The remodeling phase is underway and is converting the newly formed tissue within the wound bed into a dense irregular connective tissue that is characterized by a higher density of matrix proteins (predominantly collagens) and a lower number of capillaries. Taken together, the histological sections shown in FIG. 6 are consistent with a maturing wound that is progressing past the granulation tissue stage that is typical of chronically impaired wound healing.

D. Collagen Production

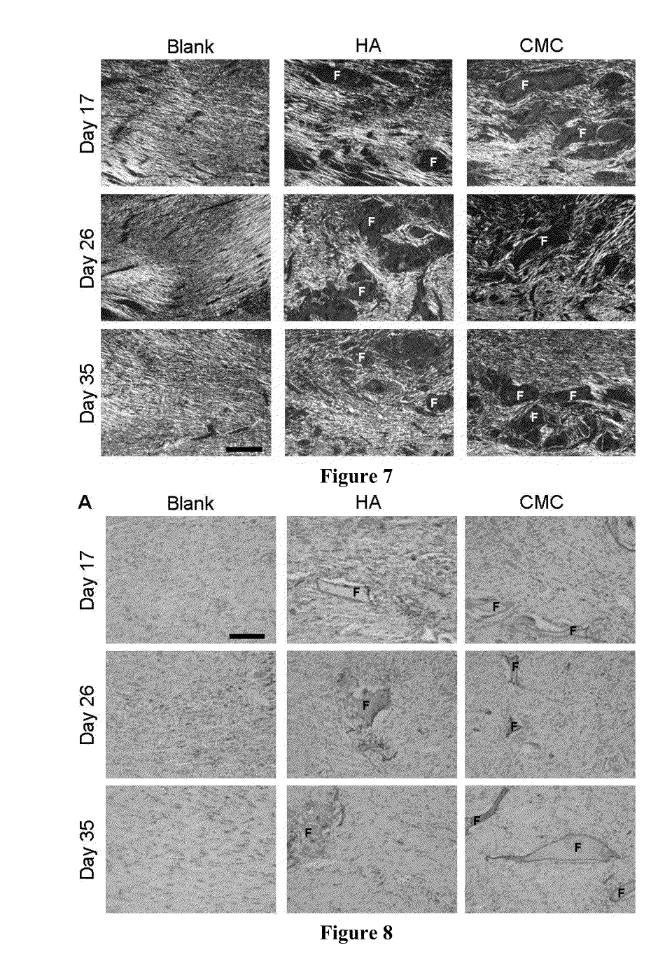

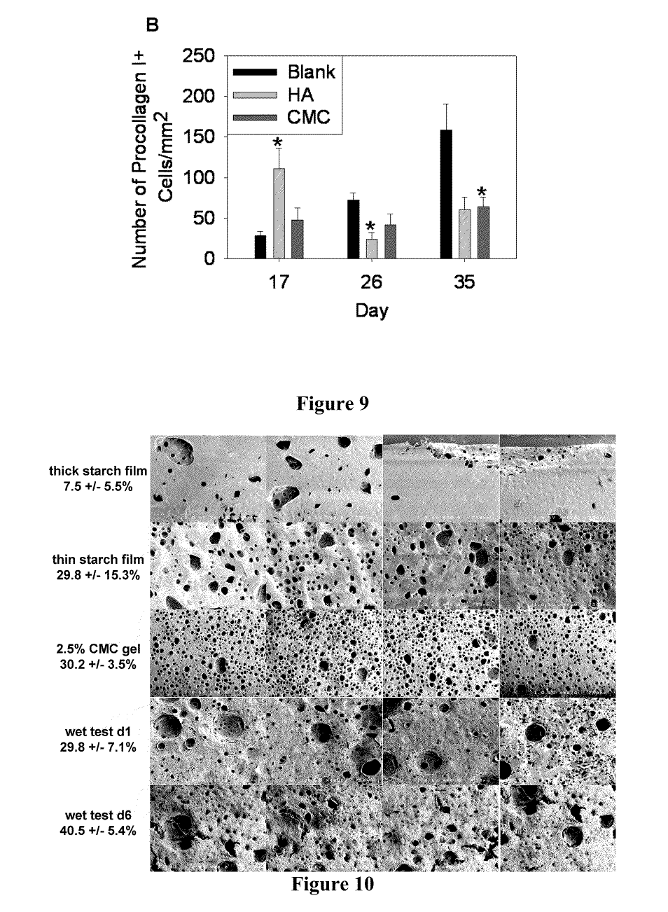

Picrosirius red staining (FIG. 7) and procollagen I (FIG. 8) immunostaining were performed in order to analyze the temporal and spatial production, accumulation, and organization of collagen in the rat excisional wounds. Picrosirius red staining shown in FIG. 7 supports the observation that collagen fiber formation in the PUR+HA and PUR+CMC treatment groups was more randomly oriented than in the blank wounds. At days 17, 26, and 35 following surgery, collagen fibers in blank wounds were organized and aligned parallel to the epidermis. In contrast, looking to shown in FIG. 8, collagen fibers surrounding polymer remnants in the HA and CMC PUR scaffolds were randomly oriented. The number of procollagen I-producing cells is quantified in FIG. 9. At day 17, there were significantly more procollagen I-producing cells in the HA group than in the blanks (p<0.02). At day 26, there were significantly fewer procollagen I-producing cells in the HA group than in the blanks (p<0.02). At day 35, there were significantly fewer procollagen I-producing cells in the CMC group than in the blanks (p<0.045).

The presence of the PUR scaffold had a modifying impact on collagen I production and deposition. Blank wounds developed a linear pattern of contraction and scarring and were highly cellular. By comparison, scaffold-treated wounds at day 35 revealed reduced cellularity and fewer collagen I secreting cells. Furthermore, the orientation of the cells and collagen fibers was more random in the presence of scaffolds. Therefore, PUR scaffolds may hinder or alter the expected scarring and contraction pattern observed in blank wounds.

### Example 8

Embodiments of composites intended for use in methods for treating wounds and that are intended to cure in situ must be able to cure in environments excess water. The following Example describes different composites and their ability to cure under “wet” conditions. To avoid undue repetition, this Example does not reiterate the materials and methods described above.

Composites that do not sufficiently cure in an aqueous environments can result in rapid degradation in wounds. This Example utilizes an in vitro wet cure test in which the materials were allowed to cure while submerged in saline. Materials were assessed to pass the test if they cured to form a solid, while materials that failed did not cure to form a solid elastomeric foam. An exemplary composite that was able to cure in saline comprises an Index of 115, 5 pphp water, 0.9 pphp DABCO 33, and 40 wt % sucrose. This exemplary composite did not comprise the blowing catalyst DMAEE, which can be cytotoxic. Using sucrose as a porogen allows for relatively higher concentrations of polysaccharide to be used in the composite.

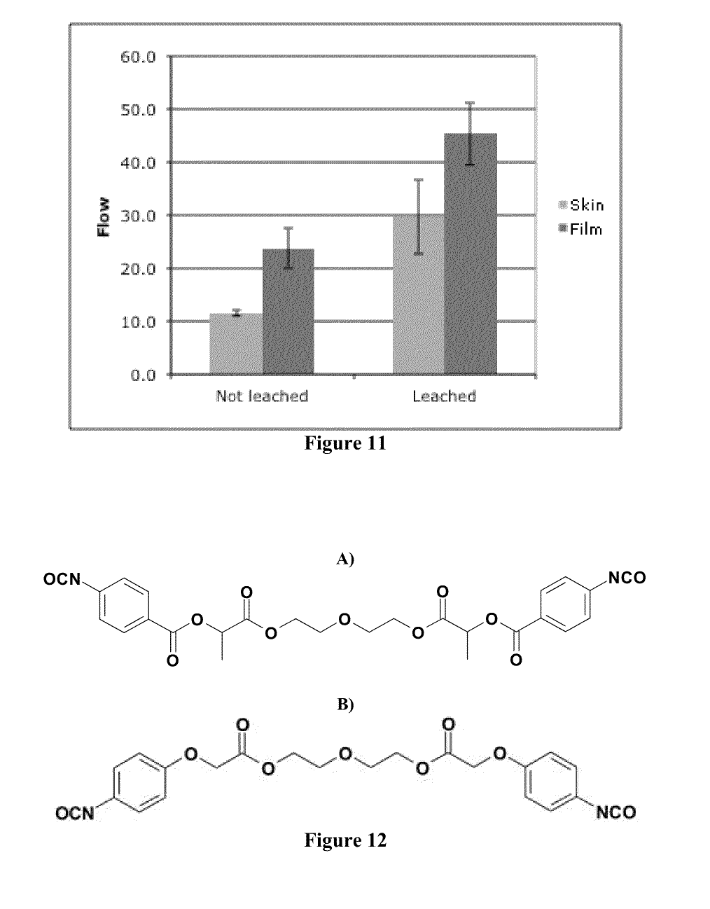

After injection, the rising foams were coated with a thick starch film, a thin starch film, or a 2.5% CMC gel. Another foam was injected directly into saline and allowed to cure (referred to as the wet test). The surface porosity of the foams was measured 24 h after cure (or 6 days in the case of one of the wet test samples). SEM images of the foams and the average porosity are presented in FIG. 10. The CMC gel resulted in the highest surface porosity and smallest pores of all the surface treatments, and was closest to the porosity observed in the wet test (40%). The air permeability of the foams was measured to assess the effects of the skin on resistance to airflow. Permeability was measured for foams that were not treated (“skin” group) and for foams treated with the 1% starch film (“film” group) to minimize skin formation before and after incubation in saline for 4 days. The results are shown in FIG. 11. Without being bound by theory or mechanism, the permeability increases after the sugar beads are leached due to the increase in porosity, and treatment with the CMC gel increases the permeability due to the increase in surface porosity.

### Example 9

This Example describes the synthesis and characterization of composites made with non-lysine triisocyanate polyisocyanates, and namely 4-para-amino benzoic acid (PABA)-lactide-diethylene glycol diisocyanate (PLD) and 4-para-amino benzoic acid (PABA)-glycolide-diethylene glycol diisocyanate (PGD). To avoid undue repetition, this Example does not reiterate the materials and methods described above.

The polyurethane foams were completed with PLD or PGD, which are shown in FIG. 12. A 3000 g/mol polyester triol soft segment was utilized for chemical crosslinking Triethylene diamine (TEDA) was utilized for all formulations based on the toxicity of DMAEE. The PLD formulation produced stable foams with a repeatable tack free time (TFT) of 12±4 minutes and initial porosities of 82±4%. The curing profile of PGD is quite similar to PLD. PGD foams are able to produce a stable product with a TFT of 15±3 minutes and initial porosities of 83±3%.

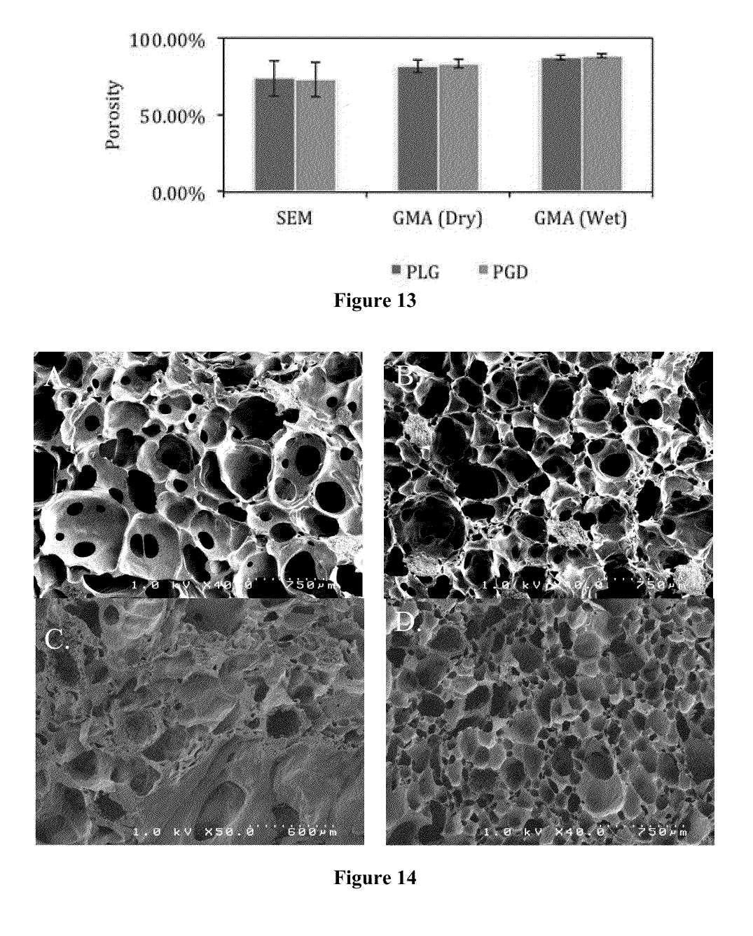

Porosity data for both PLD and PGD foams is shown in FIG. 13. Porosity was measured by gravimetric analysis (GMA) after curing for dry foams in triplicate. Foams were also injected into containers completely submerged in water to simulate an in vivo wound environment. The porosity was only increased by 5±2% with no statistical differences between the foams cured dry (FIG. 11). The difference in porosity could be due to loss of sugar during the cure.

SEM micrographs were further analyzed for cross-sectional porosity with ImageJ (FIG. 14). Pore size was also quantified via SEM and both PGD and PLD foams had porosities near 300 microns (FIG. 14A-B). The kinetics of sugar leaching was also analyzed. It was found that 80% of the sugar is leached by 48 hours and nearly all of the sugar is leached within 4 days (FIG. 14C-D). The increase in porosity ranges between 5-11%. Final porosities after leaching ranged from 90-95% for both PLD and PGD foams. Surface film formation was tested with a thick starch film (>5 mm), a solution of CMC in water, and a thin starch film (<1 mm). The foams were allowed to cure for 6 minutes, roughly half of the TFT, before the addition of either starch films or CMC. The starch films were covered with water to solubilize them directly after placing them on the foam. CMC and thick starch films produced little differences in the skin formation. The surface porosity was roughly 6-13%. Thin starch films produced the greatest reduction in skin formation, qualitatively increasing the surface porosity.

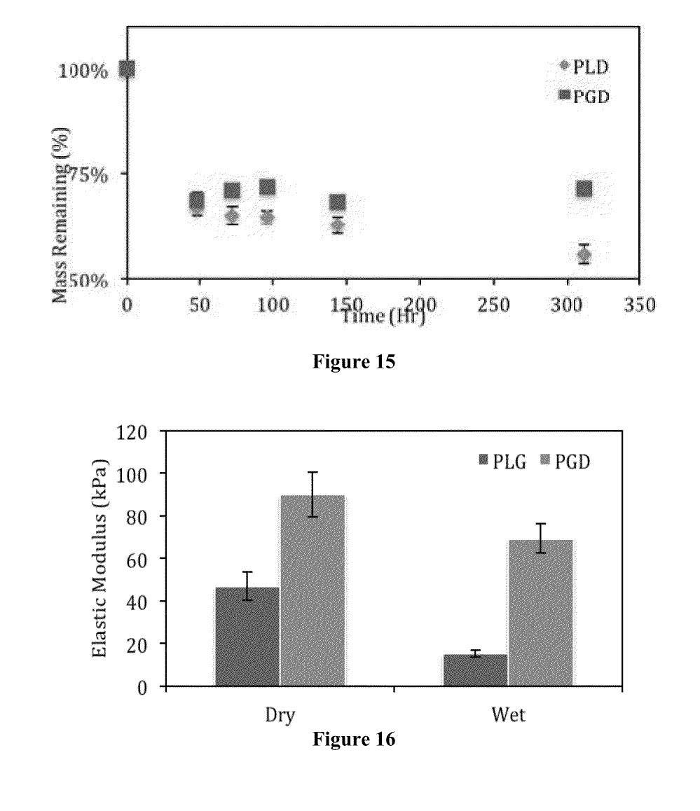

Degradation kinetics were analyzed for both PLD and PGD foams. Roughly 50-100 mg samples were placed in tubes covered in PBS in a heating block at 57° C. The samples were removed and weighed at specific intervals, shown in FIG. 15. The sugar is removed completely after 48 hours indicated by the large drop of roughly 30 wt %. After 72 hours the PLD foams began to degrade, while the PGD foams remained stable. After 6 days, the PLD foams began to disintegrate. Utilizing an activation energy of 94 kJ/mol, derived from hydrolysis of polyesters, the half-life of the PLD foams was found to be 9.9 weeks at 37° C. Over the same timeframe the PGD foams had not yet shown signs of degradation.

Mechanical testing was also completed for PLD and PGD foams in compression. Foams were tested dry and after being soaked in PBS for 7 days at 37° C. Cylindrical foam samples, roughly 11 mm in height, were analyzed with a ramp rate 1.1 mm/min following a modified version of ASTM D1621-10. Elastic modulus data was obtained, shown in FIG. 16, from the resulting stress-strain curve. PGD foams had slight decreases in elastic modulus; however, there is no statistical difference between dry and wet samples. The PLD samples are statistically different when dry and wet.

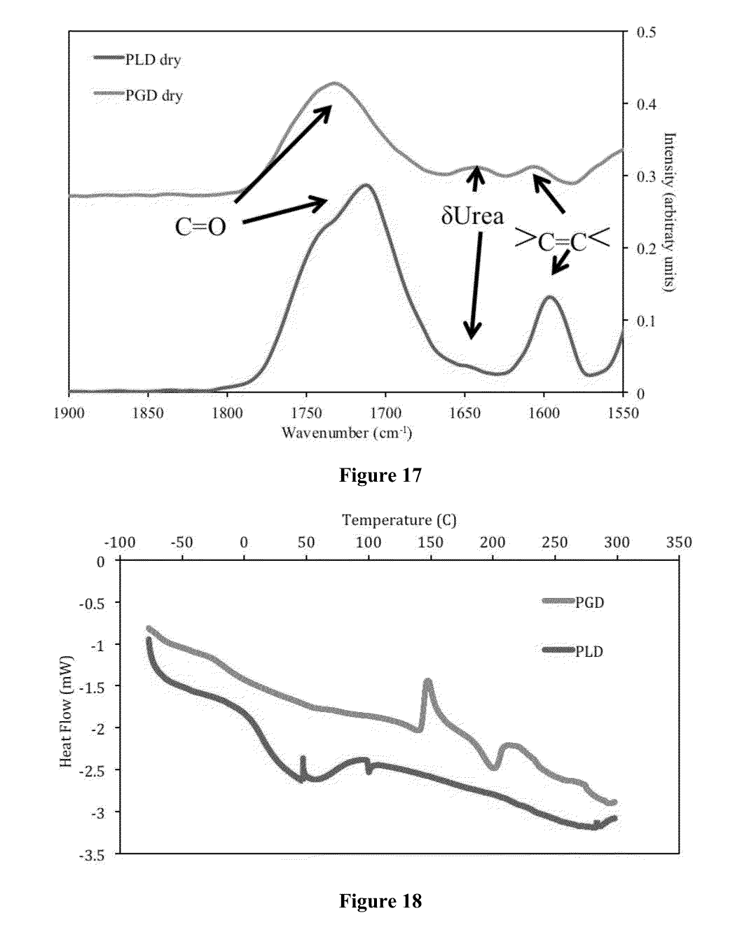

Continued analysis into the chemical structures of the foams was completed with attenuated total reflectance-Fourier transform infrared spectroscopy (ATR-FTIR) and differential scanning calorimetry (DSC). ATR-FTIR spectra were obtained from small sections of both PLD and PGD foams. The area of interest is the carbonyl region (1800-1550 cm⁻¹), shown in FIG. 17. The three peaks of interest are the urethane carbonyl (1730 for PGD and 1730-1710 for PLD); the bidentate, hydrogen bonded urea (1645 cm⁻¹ for both PLD and PGD); and the carbon-carbon stretching from the benzene ring in the isocyanate (1600 cm⁻¹ for both PLD and PGD). It was observed that the PGD foams had a larger presence of bidentate urea relative to the carbon-carbon peak than the PLD foams. Without being bound by theory or mechanism, this points to the fact that the hard segments in the PGD foams undergo significant hydrogen bonding while the PLD foams do not.

To further analyze the extent of hydrogen bonding DSC spectra were obtained for both PLD and PGD foams. 5-10 mg foams were heated to 120° C. then cooled to −80° C. The second heat ramp was utilized to obtain glass transition data. The DSC scans are shown in FIG. 18 and Table 3 displays the relevant thermal transitions.

TABLE 3
Transition temperatures derived from DSC scans of PGD
and PLD foams.

| Sample | T_{g1} (C.°) | T_{g2} (C.°) | T_{C} (C.°) | T_{M} (C.°) |
|---|---|---|---|---|
| PLD Foam | 14.8 | — | — | — |
| PGD Foam | −16.5 | 99.3 | 147.4 | 200.1 |
| Polyester Polyol | −45.9 | — | — | — |

### Example 10

This Example describes the synthesis and characterization of composites made with lysine triisocyanate and polyethylene glycol prepolymers that that include sucrose beads. To avoid undue repetition, this Example does not reiterate the materials and methods described above.

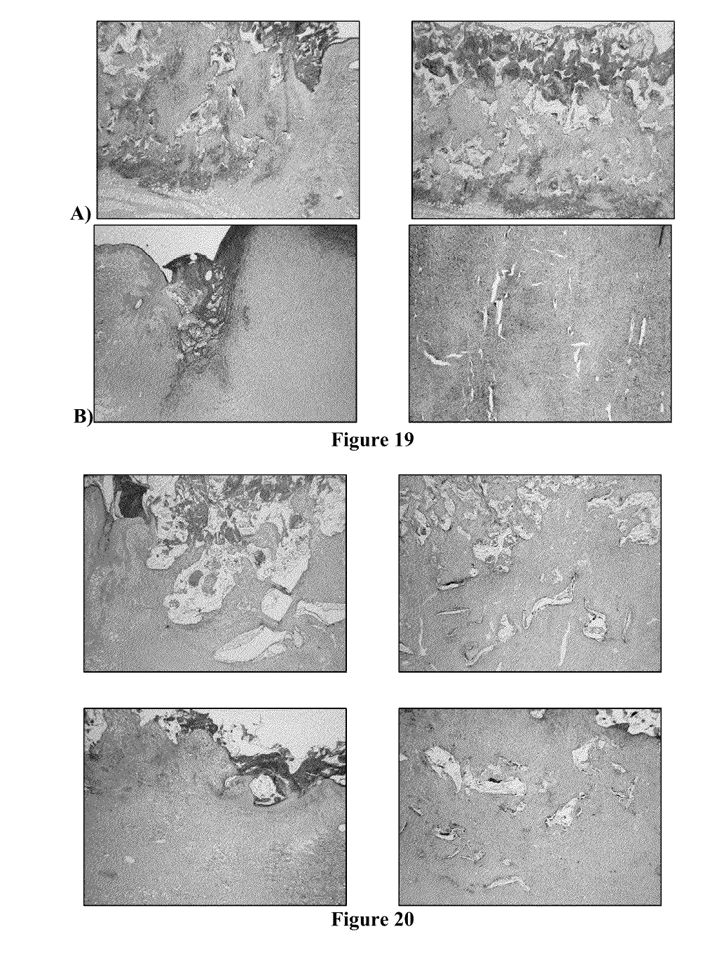

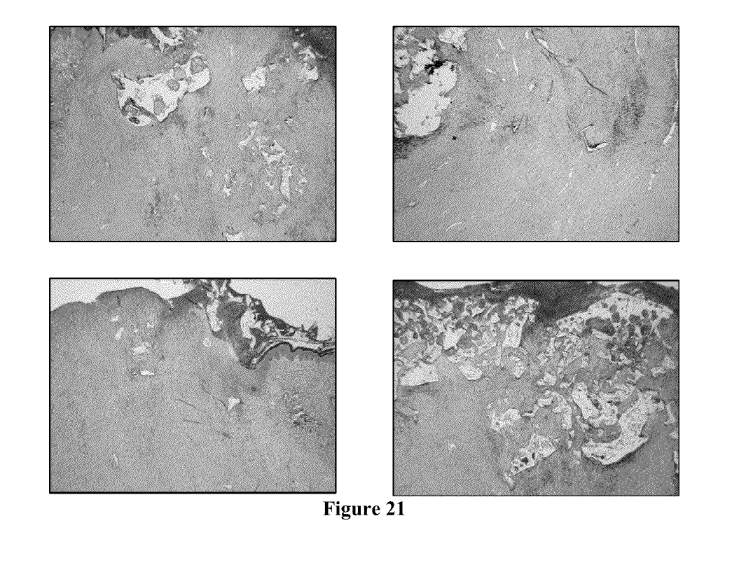

The tested composites comprised about 0.9 pphp TEDA, 5 pphp water, and had a tack free time of about 13 or 14 minutes. Furthermore, before or during the foaming process sucrose beads were added. Different composites comprised 0% sucrose (control), 40 wt % sucrose, or 70 wt % sucrose. FIGS. 19-21 shows histology from pig excisional wounds at 8 days following treatment with different scaffolds. FIG. 19 shows histological sections from pigs treated with A) a blank LTI-PEG scaffold or B) without any treatment. FIGS. 20 and 21 show histological sections from pigs treated with polyurethane composites including 40 wt % and 70 wt % of sucrose, respectively.

The invention thus being described, it will be apparent to those skilled in the art that various modifications and variations can be made in the present invention without departing from the scope or spirit of the invention. Likewise, embodiments may be practiced with all, part, or any suitable combination of the elements of the various embodiments described. Other embodiments of the invention will be apparent to those skilled in the art from consideration of the specification and practice of the invention disclosed herein. To best describe embodiments, certain structures, components, or steps well known to those skilled in the art may be lacking in this description. It is intended that the Specification, including the Example, be considered as exemplary only, and not intended to limit the scope and spirit of the invention.

Throughout this application, various publications are referenced. All such references, specifically including those listed below, are incorporated herein by reference.

### REFERENCES

1. Praemer A, Furner S, Rice D. Musculoskeletal conditions in the United States. American Academy of Orthopaedic Surgeons. Park Ridge, Ill.; 1992. p 85-124.
2. Deramond H, Wright N T, Belkoff S M. Temperature elevation caused by bone cement polymerization during vertebroplasty. Bone 1999; 25(2 Suppl):17S-21S.
3. Padovani B, Kasriel O, Brunner P, Peretti-Viton P. Pulmonary embolism caused by acrylic cement: a rare complication of percutaneous vertebroplasty. AJNR Am J Neuroradiol 1999; 20(3):375-7.
4. Khan Y, Yaszemski M J, Mikos A G, Laurencin C T. Tissue engineering of bone: Material and matrix considerations. Journal of Bone & Joint Surgery: American Edition 2008; 90(Supplement 1):36-42.
5. Augst A D, Kong H-J, Mooney D J. Alginate Hydrogels as Biomaterials. Macromolecular Bioscience 2006; 6(8):623-633.
6. Chen R R, Mooney D J. Polymeric growth factor delivery strategies for tissue engineering. Pharmaceutical Research 2003; 20(8):1103-1112.
7. Kipshidze N, Chawla P, Keelan M H. Fibrin Meshwork as a Carrier for Delivery of Angiogenic Growth Factors in Patients With Ischemic Limb. Mayo Clinic Proceedings 1999; 74(8):847-848.
8. Labhasetwar V, Bonadio J, Goldstein S, Chen W, Levy R J. A DNA controlled-release coating for gene transfer: Transfection in skeletal and cardiac muscle. Journal of Pharmaceutical Sciences 1998; 87(11):1347-1350.
9. Mizuno K, Yamamura K, Yano K, Osada T, Saeki S, Takimoto N, Sakurai T, Nimura Y. Effect of chitosan film containing basic fibroblast growth factor on wound healing in genetically diabetic mice. Journal of Biomedical Materials Research Part A 2003; 64A(1):177-181.
10. Simmons C A, Alsberg E, Hsiong S, Kim W J, Mooney D J. Dual growth factor delivery and controlled scaffold degradation enhance in vivo bone formation by transplanted bone marrow stromal cells. Bone 2004; 35(2):562-569.
11. Zawko S A, Truong Q, Schmidt C E. Drug-binding hydrogels of hyaluronic acid functionalized with β-cyclodextrin. Journal of Biomedical Materials Research Part A 2008; 87(4):1044-1052.
12. Raeber G P, Lutolf M P, Hubbell J A. Molecularly engineered PEG hydrogels: a novel model system for proteolytically mediated cell migration. Biophys J 2005; 89(2):1374-88.
13. Dumas J E, Zienkiewicz K, Tanner S A, Prieto E M, Bhattacharyya S, Guelcher S. Synthesis and Characterization of an Injectable Allograft Bone/polymer Composite Bone Void Filler with Tunable Mechanical Properties. Tissue Eng Part A 2010; 16(8):2505-18.
14. Hafeman A, Li B, Yoshii T, Zienkiewicz K, Davidson J, Guelcher S. Injectable biodegradable polyurethane scaffolds with release of platelet-derived growth factor for tissue repair and regeneration. Pharm Res 2008; 25(10):2387-99.
15. Li B, Davidson J M, Guelcher S A. The effect of the local delivery of platelet-derived growth factor from reactive two-component polyurethane scaffolds on the healing in rat skin excisional wounds. Biomaterials 2009; 30(20):3486-94.
16. Guelcher S A, Patel V, Gallagher K M, Connolly S, Didier J E, Doctor J S, Hollinger J O. Synthesis and in vitro biocompatibility of injectable polyurethane foam scaffolds. Tissue Engineering 2006; 12(5):1247-1259.
17. Hafeman A E, Zienkiewicz K J, Zachman A L, Sung H J, Nanney L B, Davidson J M, Guelcher S A. Characterization of the degradation mechanisms of lysine-derived aliphatic poly(ester urethane) scaffolds. Biomaterials 2011; 32(2):419-29.
18. Hafeman A E L B, Yoshii T, Zienkiewicz K, Davidson J M, Guelcher S A. Injectable biodegradable polyurethane scaffolds with release of platelet-derived growth factor for tissue repair and regeneration. Pharmaceutical Research 2008; 25(10):2387-2399.
19. Szycher M. Szycher's Handbook of Polyurethanes. Boca Raton: CRC Press; 1999.
20. Guelcher S, Srinivasan A, Hafeman A, Gallagher K, Doctor J, Khetan S, McBride S, Hollinger J. Synthesis, In vitro degradation, and mechanical properties of two-component poly(ester urethane)urea scaffolds: Effects of water and polyol composition. Tissue Engineering 2007; 13(9):2321-2333.
21. Kretlow J D, Klouda L, Mikos A G. Injectable matrices and scaffolds for drug delivery in tissue engineering. Advanced Drug Delivery Reviews 2007; 59:263-273.
22. Khan Y, Yaszemski M J, Mikos A G, Laurencin C T. Tissue engineering of bone: material and matrix considerations. J Bone Joint Surg Am 2008; 90 Suppl 1:36-42.
23. Kim C W, Talac R, Lu L, Moore M J, Currier B L, Yaszemski M J. Characterization of porous injectable poly-(propylene fumarate)-based bone graft substitute. J Biomed Mater Res A 2008; 85(4):1114-9.
24. Zhong S P, Zhang Y Z, Lim C T. Tissue scaffolds for skin wound healing and dermal reconstruction. Wiley Interdiscip Rev Nanomed Nanobiotechnol 2010; 2(5):510-25.
25. Lawrence B J, Madihally S V. Cell colonization in degradable 3D porous matrices. Cell Adh Migr 2008; 2(1):9-16.
26. Wang H, Pieper J, Peters F, van Blitterswijk C A, Lamme E N. Synthetic scaffold morphology controls human dermal connective tissue formation. J Biomed Mater Res A 2005; 74(4):523-32.
27. Adekogbe I, Ghanem A. Fabrication and characterization of DTBP-crosslinked chitosan scaffolds for skin tissue engineering. Biomaterials 2005; 26(35):7241-50.
28. Yang J, Shi G, Bei J, Wang S, Cao Y, Shang Q, Yang G, Wang W. Fabrication and surface modification of macroporous poly(L-lactic acid) and poly(L-lactic-co-glycolic acid) (70/30) cell scaffolds for human skin fibroblast cell culture. J Biomed Mater Res 2002; 62(3):438-46.
29. Jin Q, Wei G, Lin Z, Sugai J V, Lynch S E, Ma P X, Giannobile W V. Nanofibrous Scaffolds Incorporating PDGF-BB Microspheres Induce Chemokine Expression and Tissue Neogenesis In Vivo. PLoS ONE 2008; 3(3):e1729.
30. Scherer S S, Pietramaggiori G, Matthews J, Perry S, Assmann A, Carothers A, Demcheva M, Muise-Helmericks R C, Seth A, Vournakis J N and others. Poly-N-acetyl glucosamine nanofibers: a new bioactive material to enhance diabetic wound healing by cell migration and angiogenesis. Ann Surg 2009; 250(2):322-30.
31. Kavlock K D, Pechar T W, Hollinger J O, Guelcher S A, Goldstein A S. Synthesis and characterization of segmented poly(esterurethane urea) (PEUUR) elastomers for bone tissue engineering. Acta Biomater 2007; 3:475-484.
32. Murphy C M, Haugh M G, O'Brien F J. The effect of mean pore size on cell attachment, proliferation and migration in collagen-glycosaminoglycan scaffolds for bone tissue engineering. Biomaterials 2009; 31(3):461-6.
33. Hafeman A E, Zienkiewicz K J, Carney E, Litzner B, Stratton C, Wenke J C, Guelcher S A. Local delivery of tobramycin from injectable biodegradable polyurethane scaffolds. J Biomater Sci Polym Ed 2010; 21(1):95-112.
34. Guelcher S A, Srinivasan A, Dumas J E, et al. Synthesis, mechanical properties, biocompatibility, and biodegradation of polyurethane networks from lysine polyisocyanates. Biomaterials 2008; 29:1762-75.
35. Zhang J-Y, Beckman E J, Hu J, Yuang G-G, Agarwal S, Hollinger J O. Synthesis, biodegradability, and biocompatibility of lysine diisocyanate-glucose polymers. Tissue Eng 2002; 8(5):771-785.
36. Adhikari R, Gunatillake P A, Griffiths I, Tatai L, Wickramaratna M, Houshyar S, Moore T, Mayadunne R T, Field J, McGee M and others. Biodegradable injectable polyurethanes: synthesis and evaluation for orthopaedic applications. Biomaterials 2008; 29(28):3762-70.
37. Pailler-Matteia C, Beca S, Zahouani Z. In vivo measurements of the elastic mechanical properties of human skin by indentation tests Medical Eng and Physics 2007; In Press.
38. Seare W J, Jr. Alloplasts and biointegration. J Endourol 2000; 14(1):9-17.
39. Abramo A C, De Oliveira V R, Ledo-Silva M C, De Oliveira E L. How texture-inducing contraction vectors affect the fibrous capsule shrinkage around breasts implants? Aesthetic Plast Surg 2010; 34(5):555-60.
40. Hinz B. The myofibroblast: paradigm for a mechanically active cell. J Biomech 2010; 43(1):146-55.
41. Li B, Brown K V, Wenke J C, Guelcher S A. Sustained release of vancomycin from polyurethane scaffolds inhibits infection of bone wounds in a rat femoral segmental defect model. J Control Release 2010; 145:221-230.

We claim:

1. A composite, comprising:
a NCO-terminated prepolymer including a polyisocyanate and a first polyol;
a second polyol; and
a tissue component.

2. The composite of claim 1, wherein the tissue component comprises a polysaccharide.

3. The composite of claim 1, wherein the tissue component includes glucose, fructose, galactose, mannose, arabinose, ribose, xylose, sucrose, maltose, cellobiose, and lactose, raffinose, stachyose, starch, glycogen, cellulose, hyaluronic acid, chitosan, alginate, carboxylmethyl cellulose, or combinations thereof.

4. The composite of claim 1, comprising at least about 20 wt % of the tissue component.

5. The composite of claim 1, comprising at least about 30 vol % of the tissue component.

6. The composite of claim 1, wherein the second polyol comprises poly(caprolactone), poly(lactide), poly(glycolide), or combinations thereof.

7. The composite of claim 1, further comprising a porogen.

8. The composite of claim 1, further comprising a bioactive agent.

9. The composite of claim 8, wherein the bioactive agent is selected from the group consisting of antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, and penetration enhancer.

10. The composite of claim 1, wherein a porosity of the composite is at least about 30%.

11. The composite of claim 1, wherein the composite includes pores having a pore size of about 100 μm to about 700 μm.

12. The composite of claim 1, wherein the second polyol is a polyester polyol.

13. The composite of claim 1, wherein the polyisocyanate comprises lysine triisocyanate (LTI).

14. The composite of claim 1, wherein the first polyol comprises PEG.

15. The composite of claim 1, wherein the first polyol is the same as the second polyol.

16. The composite of claim 1, further comprising a catalyst.

17. A method of treating a wound of a subject, comprising:
administering to a wound a composite including a NCO-terminated prepolymer including a polyisocyanate and a first polyol, a second polyol, and a tissue component.

18. The method of claim 17, wherein the tissue component comprises a polysaccharide.

19. The method of claim 17, wherein the tissue component includes glucose, fructose, galactose, mannose, arabinose, ribose, xylose, sucrose, maltose, cellobiose, and lactose, raffinose, stachyose, starch, glycogen, cellulose, hyaluronic acid, chitosan, alginate, carboxylmethyl cellulose, or combinations thereof.

20. The method of claim 17, wherein the composite comprises at least about 20 wt % of the tissue component.

21. The method of claim 17, wherein the composite comprises at least about 30 vol % of the tissue component.

22. The method of claim 17, wherein the second polyol comprises poly(caprolactone), poly(lactide), poly(glycolide), and/or combinations thereof.

23. The method of claim 17, wherein the composite further comprises a bioactive agent.

24. The method of claim 23, wherein the bioactive agent is selected from the group consisting of antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, and penetraction enhancer.

25. The method of claim 17, wherein a porosity of the composite is at least about 30%.

26. The method of claim 17, wherein the composite includes pores having a pore size of about 100 μm to about 700 μm.

27. The method of claim 17, wherein the second polyol is a polyester polyol.

28. The method of claim 17, wherein the polyisocyanate comprises lysine triisocyanate (LTI).

29. The method of claim 17, wherein the first polyol comprises PEG.

30. The method of claim 17, wherein the first polyol is the same as the second polyol.

31. The method of claim 17, wherein the step of administering includes injecting the composite on to the wound and allowing the composite to cure on the wound.

32. The method of claim 17, wherein the step of administering includes molding the composite and then placing the molded composite on to the wound.

33. The method of claim 17, wherein the wound is a cutaneous wound.

34. The method of claim 17, wherein the wound is on subdermal tissue, breast tissue, vascular tissue, cardiac tissue, urogential-renal tissue, pulmonary tissue, hepatic tissue, gastrointestinal tissue, muscle tissue, ligament tissue, tendon tissue, facial tissue, gynecologic tissue, female reproductive genital tissue, non-articular surface fibrocartilage tissue, ad cartilage tissue, special sensory tissue, neural tissue, or combinations thereof.

35. A method of preparing a composite, comprising:
providing a composition that comprises a second polyol, a catalyst and water;
contacting the composition with a NCO-terminated prepolymer that includes a polyisocyanate and a first polyol;
adding at least 20 wt % of a tissue component to the composition.

36. The method of claim 5, wherein the tissue component comprises a polysaccharide.

37. The method of claim 36, wherein the tissue component includes glucose, fructose, galactose, mannose, arabinose, ribose, xylose, sucrose, maltose, cellobiose, and lactose, raffinose, stachyose, starch, glycogen, cellulose, hyaluronic acid, chitosan, alginate, carboxylmethyl cellulose, or combinations thereof.
